(19) United States
(12) Patent Application Publication
(10) Pub. No.: US 2023/0270672 A1
(43) Pub. Date: Aug. 31, 2023

(54) MICROEMULSION DRUG DELIVERY SYSTEM FOR TREATMENT OF ACUTE RESPIRATORY DISTRESS SYNDROME

(71) Applicant: Council for Scientific and Industrial Research, Pretoria (ZA)

(72) Inventors: Tshepo Patric Nkuna, Pretoria (ZA); Michel Lonji Kalombo, Pretoria (ZA); Yolandy Lemmer, Krugersdorp (ZA)

(73) Assignee: Council for Scientific and Industrial Research, Pretoria (ZA)

(21) Appl. No.: 18/007,368

(22) PCT Filed: Aug. 2, 2021
(86) PCT No.: PCT/IB2021/057057

§ 371 Date: Jan. 30, 2023

(30) Foreign Application Priority Data
Aug. 7, 2020 (ZA) 2020/04916
Aug. 7, 2020 (ZA) 2020/04917
Aug. 7, 2020 (ZA) 2020/04918

(51) Int. Cl. A61K 9/107; A61K 31/513; A61K 31/7068; A61K 31/4706; A61K 31/00; A61K 38/17

(52) U.S. Cl. CPC A61K 9/1075; A61K 31/513; A61K 31/7068; A61K 31/4706; A61K 31/658; A61K 38/1732

(57) ABSTRACT

The current invention relates to a polymer-lipid microemulsion delivery system for drugs or antiviral compounds used in the treatment or inhibition of viral Acute Respiratory Distress Syndromes (ARDS), a process for producing the microemulsion delivery system, and to methods of use of the microemulsion delivery system for the treatment of ARDS.

### FIELD OF THE INVENTION

The current invention relates to a polymer-lipid microemulsion delivery system for drugs or antiviral compounds used in the treatment or inhibition of viral Acute Respiratory Distress Syndromes (ARDS), a process for producing the microemulsion delivery system, and to methods of use of the microemulsion delivery system for the treatment of ARDS.

### BACKGROUND OF THE INVENTION

The novel coronavirus disease 2019 (COVID-19) has brought the entire global community to its knees and threatens the health and economic stability of all. It is a deadly, infectious disease caused by the severe acute respiratory syndrome coronavirus 2 (SARS-CoV-2).

However, SARS-CoV-2 is merely the most recent in a succession of pathogens resulting in respiratory illness including other severe acute respiratory syndrome coronaviruses (SARS-CoV) such as Middle East respiratory syndrome coronavirus (MERS-CoV), and the influenza viruses.

A number of institutions around the globe are in the process of developing a vaccine against SARS-CoV-2, but the development of a vaccine is a lengthy process and is costly.

There are, however, drugs that are currently in use for other syndromes and infections which have been shown to be effective or partially effective against SARS-CoV-2 in vitro and in vivo. These include Remdesivir, Lopinavir and Emtricitabine. However, Remdesivir and lopinavir are highly hydrophobic and have very poor solubility in a polar medium (0.339 mg/mL and 0.00192 mg/mL respectively). Emtricitabine, on the other hand is an acidic hydrophilic molecule, which complicates any possible co-delivery strategy with these drugs. Furthermore, fat appears to interfere with dissolution of Emtricitabine, and when taken after eating, where the gastric pH is reduced, this may further delay dissolution of orally formulated drug, resulting in lower Emtricitabine bioavailability. These drugs also have a very short half-life and are quickly metabolized and rapidly excreted from the body, resulting in high doses and frequencies of dosage (daily) being required. The approved drug Remdesivir is also expensive and in short supply and is required to be delivered by intravenous injection (IV injection) by trained personnel.

A small number of systems for pulmonary delivery of drugs are known. For example, WO 2016/030524 describes an inhalable powder formulation of alginate oligomers to form spray-dried inhalable formulations for antivirals against respiratory disorders; CN111202722 A discloses a Lopinavir dry powder pharmaceutical composition for inhalation; US2020/0179287 A1 describes electrospraying of an anionic solution containing antimicrobial drugs or antiviral drugs (e.g. Lopinavir) into a cationic solution then lyophilizing to produce an inhalable dosage; and U.S. Pat. No. 7,629,331 discloses an agglomerated beta cyclodextrin sulfobutyl ether sodium salt product known as CAPTISOL for delivery of active pharmaceutical ingredients, including Remdesivir.

However these systems employ the use of a powder formulation and can only incorporate one drug into the delivery system. Powder-based systems require time to be dissolved into liquid form for liquid administration, and to degrade the encapsulating matrix to release the drug. Alternatively, where dry powder delivery systems are introduced in the respiratory tract these have a low chance of reaching the deep lung (including the alveoli), since they are similar to dust and are therefore rapidly cleared by the immune response due to irritation of the tract. Furthermore these delivery systems are also complex to prepare and formulate with the drug of choice and require the use of expensive equipment.

A safe, effective, targeted approach to deliver antiviral drugs effective in the treatment and inhibition of SARS-CoV-2 to the site of infection that does not require invasive delivery, which is easy to use and cheap would be highly beneficial (i.e. pulmonary delivery through inhalation). It would be useful if such a delivery system enabled the simultaneous co-delivery of multiple drugs, particularly where the drugs to be co-administered were a mixture of hydrophobic and hydrophilic drugs. Such a delivery system could potentially also be used for drugs used in the treatment of other respiratory syndromes and illnesses, including those caused by viral infections such as influenza virus and other SARS-CoV including MERS.

Chloroquine and Cannabidiol are immunomodulatory drugs that have been considered for the treatment or inhibition of ARDS. Chloroquine is an antimalarial immunomodulatory compound and is known to disrupt intracellular processes, such as restricting acidification in membrane bound organelles followed by alkalizing the environment, which results in lowered or desensitized functionality of transmembrane receptors. Cannabidiol acts as a receptor binding competitor and/or a negative allosteric modulator which restricts the fusion of virus to the host cell membrane through altering or changing the receptor's affinity towards certain ligands or stimuli.

Antiviral lectins have been shown to inhibit several enveloped viruses, including lentiviruses such as human immunodeficiency virus (H IV), influenza virus and SARS-CoV by binding to mannose-rich glycans on the surface proteins of the viruses, thereby inhibiting fusion of the virus to the host cell membrane. These include griffithsin (GRFT), cyanovirin-N (CV-N), and scytovirin (SVN), more preferably GRFT and CV-N. These lectins have typically been developed for mucosal delivery through formulation in gels, creams, lubricants or suppositories, although other routes, including intravenous, intraarterial, intrathecal, intracisternal, buccal, rectal, nasal, pulmonary, transdermal, vaginal, ocular, and the like.

In the case of viral ARDS, it would be useful if such immunomodulatory compounds and fusion inhibitors could be specifically delivered to the primary site of infection by pulmonary administration. In particular, a safe, effective, targeted approach to deliver such immunomodulatory compounds effective in the treatment and inhibition of SARS-CoV-2 to the site of infection, which is easy to use and cheap, would be highly beneficial (i.e. pulmonary delivery through inhalation). It would be useful if such a delivery system enabled the simultaneous co-delivery of one or more immunomodulatory compounds, fusion inhibitors, and/or antiviral drugs, particularly where the compounds to be co-administered were a mixture of hydrophobic and hydrophilic compounds. Such a delivery system could potentially also be used for the treatment and inhibition of other respiratory syndromes and illnesses caused by viral infections such as influenza virus and other SARS-CoV, including MERS.

### SUMMARY OF THE INVENTION

According to a first aspect of the invention, there is provided a polymer-lipid microemulsion drug delivery system for the treatment or inhibition of viral Acute Respiratory Distress Syndromes (ARDS) comprising or consisting of:
i. an inner microemulsion matrix comprised or consisting of at least one fatty acid dissolved in a polar aprotic solvent, and a surfactant;
ii. an outer shell comprising or consisting one or more hydrophilic polymers; and
iii. one or more drug(s) selected from the group consisting of:
   a. antiviral drug(s);
   b. immunomodulatory compound(s); and
   c. antiviral lectin(s),
      wherein where the one or more drug(s) is a hydrophobic drug, the drug is comprised in the inner microemulsion matrix, and wherein the drug is a hydrophilic drug, the antiviral drug is comprised in the outer shell.

The one or more antiviral drug(s) may be selected from hydrophobic antiviral drugs Remdesivir and Lopinavir, and hydrophilic antiviral drug Emtricitabine.

The one or more hydrophobic immunomodulatory compound(s) may be cannabidiol (CBD) and the hydrophilic immunomodulatory compound may be chloroquine or chloroquine diphosphate.

The one or more antiviral lectin(s) may be selected from hydrophilic antiviral lectins griffithsin (GRFT), cyanovirin-N (CV-N), and scytovirin (SVN). Preferably, the antiviral lectins may be GRFT and CV-N.

The outer shell may, in particular, comprise or consist of an aqueous solution of an aqueous mixture of hydrophilic polymers such as polyvinyl alcohol (PVA) and polyethylene glycol (PEG), for example, PEG 4000.

The inner microemulsion matrix may further comprise at least one organic carboxylic acid. The at least one organic carboxylic acid may be a weak acid, including those approved for human consumption comprising acetic acid, lactic acid, citric acid, or phosphoric acid, preferably acetic acid.

Additionally, the inner microemulsion matrix may comprise at least one copolymer, poly(lactic-co-glycolic acid) or PLGA, or alternatively, any biocompatible and biodegradable polymer suitable for use in active compound or drug delivery, including polylactic acid, polyglycolic acid, or poly ϵ-caprolactone.

Preferably the at least one fatty acid comprises or consists of any one or more of stearic acid, palmitic acid and lauric acid, preferably stearic acid.

The polar aprotic solvent may comprise of either ethanol or acetone, or may be a blend of ethanol and acetone. Preferably the polar aprotic solvent is acetone.

The surfactant may comprise any surfactant having a Hydrophile-Lipophile Balance (HLB) value of greater than 10. Preferably, the surfactant is polysorbate 80, also known as Tween 80®.

The microemulsion is defined as a thermodynamically stable water-in-oil or oil-in-water emulsion stabilised by a blend of surfactants and co-surfactants that is formed spontaneously with minimal input of mechanical energy. This is in contrast with other types of emulsions, so called kinetically stable emulsions, which require high shear input for them to form.

The microemulsion of the invention is typically isotropic and translucent owing to the small droplet size of the dispersed phase which ranges below about 150 nm.

The viral ARDS may be SARS-CoV, including SARS-CoV-2 and MERS-CoV, or influenza. Preferably, the viral ARDS is SARS-CoV-2.

According to a further aspect of the invention, there is provided a process for producing a polymer-lipid microemulsion drug delivery system comprising one or more drug(s) selected from the group consisting of antiviral drug(s); immunomodulatory compound(s); and antiviral lectin(s), comprising or consisting essentially of the steps of:
A.I. mixing at least one hydrophobic drug, a fatty acid dissolved in a polar aprotic solvent, and a surfactant to form an organic phase;
A.II. optionally heating the organic phase;
A.III. dispensing the organic phase into an aqueous mixture comprising at least one hydrophilic polymer to form a microemulsion; and
A.IV. stabilising the microemulsion in a phosphate buffer at about 0° C. to form the polymer-lipid microemulsion, or
B.I. mixing a fatty acid dissolved in a polar aprotic solvent, and a surfactant to form an organic phase;
B.II. optionally heating the organic phase;
B.III. dispensing the organic phase into an aqueous mixture comprising at least one hydrophilic polymer and at least one hydrophilic drug to form a microemulsion; and
B.IV. stabilising the microemulsion in a phosphate buffer at about 0° C. to form the polymer-lipid microemulsion, or
C.I. mixing at least one hydrophobic drug, a fatty acid dissolved in a polar aprotic solvent, and a surfactant to form an organic phase;
C.II. optionally heating the organic phase;
C.III. dispensing the organic phase into an aqueous mixture comprising at least one hydrophilic polymer and at least one hydrophilic drug to form a microemulsion; and
C.IV. stabilising the microemulsion in a phosphate buffer at about 0° C. to about 10° C. form the polymer-lipid microemulsion.

The one or more antiviral drug(s) may be selected from hydrophobic antiviral drugs Remdesivir and Lopinavir, and hydrophilic antiviral drug Emtricitabine.

The one or more hydrophobic immunomodulatory compound(s) may be cannabidiol (CBD) and the hydrophilic immunomodulatory compound may be chloroquine or chloroquine diphosphate.

The one or more antiviral lectin(s) may be selected from hydrophilic antiviral lectins griffithsin (GRFT), cyanovirin-N (CV-N), and scytovirin (SVN). Preferably, the antiviral lectins may be GRFT and CV-N.

The polymer-lipid microemulsion delivery system may be a liquid and may be nebulised for delivery by inhalation, including for pulmonary delivery.

The process may optionally further comprise a final step of drying the stabilised polymer-lipid microemulsion to produce a free flowing polymer-lipid microemulsion powder either by freeze drying or by spray drying. The free flowing polymer-lipid microemulsion delivery system may be formulated for oral or intravenous delivery.

The process may further comprise mixing an organic carboxylic acid with the organic phase.

The process may further comprise dissolving at least one biocompatible and biodegradable polymer or copolymer suitable for use in active compound delivery, poly(lactic-co-glycolic acid) or PLGA, or polylactic acid, polyglycolic acid, or poly ϵ-caprolactone, into the polar aprotic solvent with the fatty acid to form the organic phase.

The at least one fatty acid may comprise or consist of any one or more of stearic acid, palmitic acid and lauric acid, preferably stearic acid.

The polar aprotic solvent may comprise either ethanol or acetone, or may be a blend of ethanol and acetone. Preferably the polar aprotic solvent is acetone.

The organic carboxylic acid may comprise at least one weak acid. For example, the weak acid may include any one or more of those approved for human consumption comprising acetic acid, lactic acid, citric acid, or phosphoric acid. Preferably the weak acid is acetic acid.

The surfactant may comprise any surfactant having a Hydrophile-Lipophile Balance (HLB) value of greater than 10. Preferably, the surfactant is polysorbate 80, also known as Tween 80®.

In particular, the process may comprise or consist of the following steps:
A.a) dissolving at least one fatty acid in a polar aprotic solvent to form a fatty acid solution;
A.b) dissolving at one or more hydrophobic drug(s) in the fatty acid solution;
A.c) adding drop-wise, a surfactant to form an organic phase;
A.d) optionally heating the organic phase;
A.e) dispensing the organic phase into an aqueous mixture comprising at least one hydrophilic polymer, and optionally one or more hydrophilic drug(s) while stirring to form a microemulsion; and
A.f) stabilising the polymer-lipid microemulsion by adding a phosphate buffer at 0° C. while stirring, or
B.a) dissolving at least one fatty acid in a polar aprotic solvent to form a fatty acid solution;
B.b) optionally dissolving one or more hydrophobic drug(s) in the fatty acid solution;
B.c) adding drop-wise, a surfactant to form an organic phase;
B.d) optionally heating the organic phase;
B.e) dispensing the organic phase into an aqueous mixture comprising at least one hydrophilic polymer, and one or more hydrophilic drug(s) while stirring to form a microemulsion; and
B.f) stabilising the polymer-lipid microemulsion by adding a phosphate buffer at 0° C. while stirring, or
C.a) dissolving at least one fatty acid in a polar aprotic solvent to form a fatty acid solution;
C.b) dissolving one or more hydrophobic drug(s) in the fatty acid solution;
C.c) adding drop-wise, a surfactant to form an organic phase;
C.d) optionally heating the organic phase;
C.e) dispensing the organic phase into an aqueous mixture comprising at least one hydrophilic polymer, and one or more hydrophilic drug(s) while stirring to form a microemulsion; and
C.f) stabilising the polymer-lipid microemulsion by adding a phosphate buffer at 0° C. while stirring.

The process may further comprise an additional step of drying the stabilised polymer-lipid microemulsion to produce a free flowing polymer-lipid nanocomplex powder either by freeze drying or by spray drying.

The process may further comprise, at step a), dissolving PLGA, or alternatively, any biocompatible and biodegradable polymer suitable for use in active compound delivery, including polylactic acid, polyglycolic acid, or poly ϵ-caprolactone, into the polar aprotic solvent with the fatty acid.

The process may further comprise, at step c), adding drop-wise, an organic carboxylic acid with the surfactant.

The process may further comprise in step e) heating while stirring to form the microemulsion. The heating steps may be performed at from between about 40° C. to 50° C., preferably 40° C.

The phosphate buffer may comprise a pH of from about 7.2 to about 7.6, more preferably, about 7.4 at 0° C.

The stabilisation of the microemulsion may be performed by adding the microemulsion to the phosphate buffer solution at a ratio about 1:1. It is to be appreciated that a variety of factors influence the optimum ratio of microemulsion to buffer, including drug loading, stability of the formulation, including during the drying process, and the like.

The freeze drying may be performed following an initial snap-freezing step in liquid nitrogen.

The spray drying may be performed using a spray dryer such as the Top bench Buchi-B290. In particular, such spray drying may be performed with the following set of parameters;
Inlet temperature: about 90 to 110° C.
Outlet temperature: about 60° C.
Feeding rate: 2% (mL/min)
Atomizing pressure: 6-7 bar
Aspiration vacuum set at 100%.

It is to be appreciated that the inlet temperature should be high enough to evaporate both the polar (water) and nonpolar (organic) solvents without degrading any compounds in the formulation, and that the range provided is one embodiment of the invention and may be modified by those skilled in the art.

It is further to be appreciated that outlet temperature is affected by the room temperature of the lab in which the apparatus is situated and, apart from requiring that the outlet temperature is above 60° C. in order to obtain a dry, free flowing powder, the specific temperature may vary. The outlet temperature is equally governed by the liquid feeding rate, the inlet temperature and thermal exchange efficiency between droplets and the drying hot air.

According to a further aspect of the invention, there is provided a method for the treatment or inhibition of viral ARDS with the polymer-lipid microemulsion delivery system of the invention comprising one or more drug(s) selected from the group consisting of antiviral drug(s); immunomodulatory compound(s); and antiviral lectin(s), as described above.

The viral ARDS may be SARS-CoV, including SARS-CoV-2 and MERS-CoV, or influenza. Preferably, the viral ARDS is SARS-CoV-2.

The method may comprise delivery by pulmonary administration of a liquid formulation of the polymer-lipid microemulsion delivery system of the invention.

The method may comprise delivery by oral or intravenous administration of a powder formulation of the polymer-lipid microemulsion delivery system of the invention.

The method may comprise simultaneous delivery by pulmonary administration of a liquid formulation of the polymer-lipid microemulsion delivery system of the invention and oral or intravenous administration of a powder formulation of the polymer-lipid microemulsion delivery system of the invention.

The method may comprise a step of nebulising the liquid polymer-lipid microemulsion delivery system for delivery by inhalation, including for pulmonary delivery.

### BRIEF DESCRIPTION OF THE DRAWINGS

The invention shall be described with reference to the following illustrations, which should in no way be interpreted as limiting the scope of the invention:

FIG. 1 shows the size and size distribution of Emtricitabine incorporated in the microemulsion delivery system;

FIG. 2 shows the size and size distribution of Remdesivir incorporated in the microemulsion delivery system;

FIG. 3 shows the size and size distribution of Lopinavir incorporated in the microemulsion delivery system;

FIG. 4 shows the size and size distribution of Emtricitabine and Remdesivir incorporated in the same microemulsion delivery system;

FIG. 5 shows the size and size distribution of Remdesivir incorporated in the hybrid polymer-lipid nanocomplex delivery system;

FIG. 6 shows the size and size distribution of Lopinavir incorporated in the hybrid polymer-lipid nanocomplex delivery system;

FIG. 7 shows the calibration curves of Emtricitabine, Remdesivir and Lopinavir;

FIG. 8 shows the analytical detection of drug retention peaks incorporated in delivery systems;

FIG. 9 shows the physicochemical results of delivery systems incorporating the drugs;

FIG. 10 shows a graphical illustration of the microemulsion delivery system;

FIG. 11 shows the hydrodynamic size and size distribution of CBD;

FIG. 12 shows the hydrodynamic size and size distribution of CQ;

FIG. 13 shows the hydrodynamic size and size distribution of CBD and CQ;

FIG. 14 shows the calibration curves of CBD and CQ;

FIG. 15 shows the drug loadings of CBD and CQ;

FIG. 16 shows CBD inhibiting infection of cells by the HIV-1 pseudo virus;

FIG. 17 shows CQ inhibiting infection of cells by the HIV-1 pseudo virus;

FIG. 18 shows the combination of CBD and CQ inhibiting infection of cells by the HIV-1 pseudo virus;

FIG. 19 shows the size of the microemulsion delivery system without the active compound obtained via dynamic light scattering Malvern NanoZS equipment;

FIG. 20 shows the size of a lectin-loaded microemulsion delivery system;

FIG. 21 shows a qualitative characterization by an HPLC, depicting an active antiviral lectin post-formulation, unaltered by the formulation process;

FIG. 23 shows the antiviral activity of CVN; and

FIG. 24 shows the antiviral activity of GRFT.

### DETAILED DESCRIPTION OF THE INVENTION

The present invention relates to a polymer-lipid microemulsion delivery system for one or more drugs or active compounds used in the treatment or inhibition of viral Acute Respiratory Distress Syndromes (ARDS), a process for producing the microemulsion delivery system, and to methods of use of the microemulsion delivery system for the treatment of ARDS.

Remdesivir, Lopinavir and Emtricitabine are currently in use for other syndromes and infections which have been shown to be effective or partially effective against SARS-CoV-2 in vitro and in vivo. However, Remdesivir and lopinavir are highly hydrophobic and Emtricitabine is an acidic hydrophilic molecule, which complicates any possible co-delivery strategy with these drugs.

Chloroquine and Cannabidiol are immunomodulatory drugs that have been considered for the treatment or inhibition of ARDS.

Antiviral lectins including GRFT, CV-N, and SVN have been used to inhibit virus binding to host cell by binding to mannose-rich glycans on the surface proteins of the viruses, thereby inhibiting fusion of the virus to the host cell membrane. These lectins have typically been delivered mucosally through formulation in gels, creams, lubricants or suppositories for inhibition of HIV, although other routes, including intravenous, intraarterial, intrathecal, intracisternal, buccal, rectal, nasal, pulmonary, transdermal, vaginal, ocular, and the like have also been proposed depending on the target virus.

In the case of viral ARDS it would be useful if the abovementioned drugs or active compounds could be specifically delivered to the primary site of infection by pulmonary administration. It would also be useful if the immunomodulatory compounds could, in cases of severe infection be delivered both by pulmonary administration and by intravenous and/or oral administration using the same delivery vehicle which was safe, effective, simple and cheap to produce.

Furthermore, it would be very useful to have a delivery system that provides for co-delivery of both hydrophobic and hydrophilic drug compounds such as those described above.

The applicant has therefore developed a polymer-lipid microemulsion delivery system for targeted pulmonary administration of one or more drug(s) or active compound(s) for the treatment or inhibition of viral ARDS, including those caused by SARS-CoV such as SARS-CoV-2 and MERS-CoV as well as influenza. The polymer-lipid microemulsion delivery system is versatile, in that it can either be formulated as a liquid for nebulising and pulmonary administration, or could be formulated as a free-flowing powder for oral and/or intravenous administration.

A further advantage of the polymer-lipid microemulsion system developed by the applicant is that it may be used for simultaneous co-delivery of one or more drug(s) or active compound(s), including where these are a mixture of hydrophobic and hydrophilic drug(s) or active compound(s). The delivery system can incorporate up to three drugs or active compounds with different hydrophobicities or hydrophilicities in one system.

Drugs and other active molecules that have been used in the treatment or inhibition of viral ARDS have a number of short-falls which include low absorption in the lumen, high metabolism by the liver, and severe adverse effects due to high dosages and frequencies. The delivery mechanism provided by the polymer-lipid microemulsion system of the invention addresses these issues.

The delivery system is non-invasive, safe and it is 99% water-based. When used in conjunction with hydrophobic active compounds, the polymer-lipid microemulsion system improves the solubility of hydrophobic drugs, which in turn improves absorption and bypasses the first-pass metabolism by the liver enzymes, resulting in a greater number of active compounds being available to treat viral ARDS.

Due to the targeted pulmonary delivery of the polymer-lipid microemulsion system, there is a higher deposition of antiviral drugs and compounds encapsulated therein at the primary sites of infection. This provides for the use of lower active compound doses and dosage frequencies, quicker onset of antiviral activity and reduced treatment durations.

The polymer-lipid microemulsion system has been successfully developed and inhibition activity was observed in a biological inhibition assay in vitro using an HIV pseudo-virus.

The exemplary examples below are for illustrative purposes and should in no way be construed as limiting in any way the scope of the invention.

### EXAMPLE 1

### Development of Delivery Systems Incorporating Antiviral Drugs Repurposed for the Treatment of COVID-19 (Emtricitabine, Remdesivir and Lopinavir)

1. Background

Viruses are ubiquitous and the smallest non-living organisms known to infect all types of life forms and cause disease in a diverse range of multicellular organisms. They lack key cellular characteristics such as the cell membrane and can ONLY replicate within a living host cell. Critical processes necessary for their survival depends entirely on the ability to infect a host cell and exploit its processes for replication. Briefly, viruses attach to cellular transmembrane proteins (i.e. receptors) then insert their viral genome into the host (i.e. endocytosis) and replicate to produce numerous new virions which infect other cells. Currently, there is no treatment or vaccine for the COVID-19 disease, although a few antiviral drugs have shown to be effective through the inhibition of their viral genome replication in in-vitro biological assays. These include transcription and protease inhibitors such as Emtricitabine, Remdesivir and Lopinavir.

Emtricitabine is a synthetic cytidine nucleoside analogue that is intracellularly phosphorylated to its active metabolite, emtricitabine 5′-triphosphate by cellular enzymes. It acts as a competitor with the host cytidine substrates and through its incorporation causes early chain sequence termination. Emtricitabine has also been shown to promote the increase of immune cells such as CD4⁺ T cells. Remdesivir has the same mechanism of action as emtricitabine; it was initially developed for the treatment of the Ebola virus. A recent study of remdesivir against SARS-CoV-2 showed a shortened recovery period in severe cases and was granted further use as an experimental drug. Lopinavir is an antiviral molecule approved for HIV treatment; it is a synthetic protease inhibitor that can inhibit the action of the HIV-1 protease. It has shown efficacy through blocking the 3C-like protease of the coronaviruses and is being investigated further as a potential drug to be used against the COVID-19.

2. Methods and Materials

2.1 Materials and Equipments

Emtricibine (ETB), Remdisivir (RDV) and Lapinovir (LPV) were kindly supplied by Abdi Ibrahim Hag (Istanbul, Turkey). Solvents were all purchased from Sigma and include ethanol, acetone, acetonitrile, dimethyl sulfoxide (DMSO), ethyl acetate, dichloromethane (DCM) and oleic acid. Polyvinyl alcohol (PVA) (87-89 hydrolysed/Mw=13000-23000), polyethylene glycol (PEG) (Mw—4000), stearic acid and phosphate buffer saline (PBS—pH 7.4) reagents were all obtained from Sigma Aldrich, South Africa. Phosphoric acid and trimethylamine (TEA) were purchased from Sigma Aldrich. All other chemicals and reagents were of an analytical grade.

Malvern Zetasizer nano series ZS (DLS) was used to determine the hydrodynamic size, size distribution and the stabily of the microemulsions and a Shimadzu SIL-20AXR/20ACRXR prominence High Pressure Liquid Chromatography (HPLC) for qualitative analysis. Analyses were performed on a Shimadzu SIL-20AXR/20ACRXR prominence Liquid Chromatography (HPLC) which consisted of a LC-20AT solvent delivery module equipped with SIL-20AXR/20ACXR autosampler, a SPD-M20A UV/VIS photodiode array detector set, and SN4000 LabSolutions system software. The HPLC separation was carried out using a phenomenex LUNA C18 column (150×4.6 mm id; 5 micron particle size).

2.2 Methods

2.2.1 Preparation of Microemulsions—Hydrophilic Drug

Internal/Organic Phase

Briefly, the microemulsion system with emtricitabine was prepared as follows: The internal organic phase was prepared by dissolving of PLGA (5 to 20 mg) and stearic acid (1 to 5 mg) in a co-solution of acetone/ethanol followed by the addition of 10 to 20 μl of a surfactant with a high HLB value above 10 (Tween 80°).

Continuous/Polar Phase

The continuous polar phase was prepared by mixing equal portions of one buffering solution of phosphate buffer saline (PBS pH 7.4) with two hydrophilic polymers, polyvinyl alcohol (PVA) and polyethylene glycol (i.e. PEG 4000). Emtricitabine (10-100 mg) was added to and dissolved in the continuous phase.

Emulsion Formation

To form the microemulsion, the organic phase was added to the continuous phase while stirring moderately at room temperature (23-25° C.). The spontaneous precipitation of the PLGA/SA resulted in the self-assembly of a thermodynamically stable microemulsion via nucleation. The system was then stirred under fumehood for 2 hours to evaporate the solvents. The microemulsions were transparent, stable for over 2 months and had a light blue distinct appearance of a phenomenon known as the Tyndall effect.

2.2.2 Preparation of Microemulsions—Hydrophobic Drugs

Internal/Organic Phase

For the preparation of the microemulsion with remdesivir or lopinavir, the internal phase was prepared by dissolving PLGA and stearic acid in a co-solution of acetone/ethanol. This was followed by the addition of 10 to 20 μl of a surfactant with a high HLB value above 10 (Tween 80®) to which 50-100 μl of an organic carboxylic acid, acetic acid, was added. The drug, RDV or LPV (5-20 mg) was added to and dissolved in the organic solution resulting into the oil phase (internal) of the emulsion. The dissolved drug in the oil phase may optionally be heated to about 40° C. before adding to the aqueous mixture of hydrophilic polymers, and the moderate stirring may be performed at about 40° C. on a magnetic hot plate.

Continuous/Polar Phase

The continuous polar phase was prepared by mixing equal portions of one buffering solution of phosphate buffer saline (PBS pH 7.4) with two hydrophilic polymers, polyvinyl alcohol (PVA) and polyethylene glycol (i.e. PEG 4000).

Emulsion Formation

To form the microemulsion, the organic phase was added to the continuous phase while stirring moderately at room temperature (23-25° C.). The spontaneous precipitation of the PLGA/SA resulted in the self-assembly of a thermodynamically stable microemulsion via nucleation. The system was then stirred under fumehood for 2 hours to evaporate the solvents. The microemulsions were transparent, stable for over 2 months and had a light blue distinct appearance of a phenomenon known as the Tyndall effect.

2.2.3 Preparation of microemulsions—Hydrophobic and hydrophilic drugs

Internal/Organic Phase

For the preparation of the microemulsion with remdesivir and emtricitabine, the internal organic phase was prepared by dissolving PLGA and stearic acid in a co-solution of acetone/ethanol followed by the addition of 10 to 20 μl of a surfactant with a high HLB value above 10 (Tween 80®) to which 50-100 μl of an organic carboxylic acid, acetic acid, was added. The drug, RDV (5-20 mg) was added to and dissolved in the organic solution resulting into the oil phase (internal) of the emulsion. It is also possible to optionally dissolve any biocompatible and biodegradable polymer suitable for use in drug delivery, including polylactic acid, polyglycolic acid, or poly ϵ-caprolactone, in the stearic acid and acetone/ethanol co-solution to further improve stability of the hydrophobic active in the inner matrix of the microemulsion. The organic phase may optionally first be heated to about 40° C., then, when dispensed into the aqueous mixture of hydrophilic polymers, may be moderately stirred using a magnetic hot plate at about 40° C.

Continuous/Polar Phase

The continuous polar phase was prepared by mixing equal portions of one buffering solution of phosphate buffer saline (PBS pH 7.4) with two hydrophilic polymers, polyvinyl alcohol (PVA) and polyethylene glycol (i.e. PEG 4000). Emtricitabine (10-100 mg) was added to and dissolved in the continuous phase.

Emulsion Formation

To form the liquid microemulsion, the organic phase was rapidly dispensed into continuous phase while stirring moderately at room temperature (23-25° C.). The spontaneous precipitation resulted in the self-assembly of a thermodynamically stable microemulsion via nucleation. The system was then stirred under fumehood for 2 hours to evaporate the solvents. The microemulsions were transparent, stable for over 2 months and had a light blue distinct appearance of a phenomenon known as the Tyndall effect.

2.2.4 Preparation of Nanoparticles—Hydrophobic Drugs

Internal/Organic Phase

For the preparation of the nanoparticles with remdesivir or lopinavir, the internal organic phase was prepared by dissolving PLGA and stearic acid in a co-solution of acetone/ethanol followed by the addition of 10 to 20 μl of a surfactant with a high HLB value above 10 (Tween 80®) to which 50-100 μl of an organic carboxylic acid, acetic acid, was added. Upon complete dissolvation of PLGA and stearic acid, the hydrophobic drug (100-300 mg) was added to and dissolved in the organic solution continued to stir moderately for 3-5 minutes resulting into the oil phase (internal) of the emulsion. It is also possible to optionally dissolve alternatively, any biocompatible and biodegradable polymer suitable for use in drug delivery, including polylactic acid, polyglycolic acid, or poly ϵ-caprolactone, in the stearic acid and acetone/ethanol co-solution to further improve stability of the hydrophobic active in the inner matrix of the microemulsion.

Continuous/Polar Phase

The continuous polar phase was prepared by mixing equal portions of one buffering solution of phosphate buffer saline (PBS pH 7.4) with two hydrophilic polymers, polyvinyl alcohol (PVA) and polyethylene glycol (i.e. PEG 4000).

Emulsion Formation

To form the liquid microemulsion, the organic phase was rapidly dispensed into continuous phase while stirring moderately at room temperature (23-25° C.). The spontaneous precipitation resulted in the self-assembly of a thermodynamically stable microemulsion via nucleation. The organic phase may optionally first be heated to about 40° C., then, when dispensed into the aqueous mixture of hydrophilic polymers, may be moderately stirred using a magnetic hot plate at about 40° C., resulting in a stable microemulsion with reproducible droplet size and size distribution. The resultant O/W emulsion was then added to a cold solution of phosphate buffered saline (pH7.4) to further stabilize the emulsion. The emulsion was then spray dried at 95-110° C. with an atomising pressure between 5 and 8 bars. All formulations yielded free flowing powders after the spray drying process and were readily re-dispersed in aqueous solutions resulting in translucent nanosuspensions.

2.2.6 Physicochemical Characterization

2.2.6.1 Hydrodynamic size, Size Distribution and Stability

The hydrodynamic size, size distribution and stability of the delivery systems were determined by a Dynamic Light Scattering (DLS) technique using the Malvern Zetasizer Nano series ZS. The DLS instrument measures the Brownion motion, the random movement (fluctuation) of submicrom particles in a solution to determine the hydrodynamic size. Briefly, a laser beam is used to illuminate the sample solution, the incident laser beam gets scattered in all direction and the intensity measured by a detector. To elaborate, continuous data correlation of the speed and the count rate in kilocounts per second (Kcps) of particle diffusion in solution are the key parameters for size determination. The smaller particles in solution diffuse faster than the larger particles. Stability of submicron particles can also be determined by DLS over time through continuous sample analysis. Samples for analysis for both the microemulsions and the nanoparticles were prepared in deionized water, diluted 300 to 400 times and a disposable zetasizer cuvette was used for the analysis.

2.2.6.2 Qualitative and Quantitative Analysis

The analytical methods used were developed by following the guidelines from the U.S. Department of Health and Human Services Food and Drug Administration on Analytical Procedures and Methods Validation for Drugs and Biologics, Guidance for Industry published in 2015 and European Pharmacopoeia (EP10.0). The HPLC separation was carried out using a phenomenex LUNA C₁₈ column (150×4.6 mm id; 5 micron particle size) and a mobile phase composed of methanol (A) and 0.1% aqueous triethylamine, pH 3.2 adjusted with phosphoric acid (B), at a flow rate 0.4 mL/min. The gradient elution programme was 10% A from 0-1.9 min, 10-40% A from 1.9-2.0 min and 40% A from 2.0-3.3 min. For column re-equilibration, 10% A was maintained from 3.3-5.00 min. UV detection was performed at 220-260 nm and the injection volume was 20 μl.

A simple, rapid, and selective reversed-phase high-performance liquid-chromatography (HPLC-UV) method was developed for the determination of Emtricitabine, Remdesivir and Lopinavir for both delivery systems. Calibration curves were prepared by the analysis of blank delivery system samples spiked with various concentrations of working solutions of the drug substances. The samples were then submitted to the processes such as sonication, chromatographic separation, and UV detection described above. Calibration curves were obtained by linear least-squares regression analysis plotting of peak areas versus the concentrations. The calibration curve equation is y=ax+b, where y represents the peak areas and x represents the concentrations of the drug substances. The limit of detection (LOD) was determined as the lowest concentration giving a signal to noise ratio (S/N) of 3 for all of the drug substances. Limit of quantification (LOQ), the lowest amount of analyte that can be quantified with acceptable precision and accuracy, was determined as S/N of 10.

Stock solutions of the drugs were prepared in methanol/water (50:50). Prior to measurements, stock solutions were diluted with methanol-water (50:50, v/v) so as to prepare the working standard solutions of 100 μg/mL and 1 μg/mL. Various dilutions were made to prepare working solutions. HPLC analysis was carried out with 20 μL aliquots of various concentrations of the working solutions.

3. Results

3.1 Delivery System

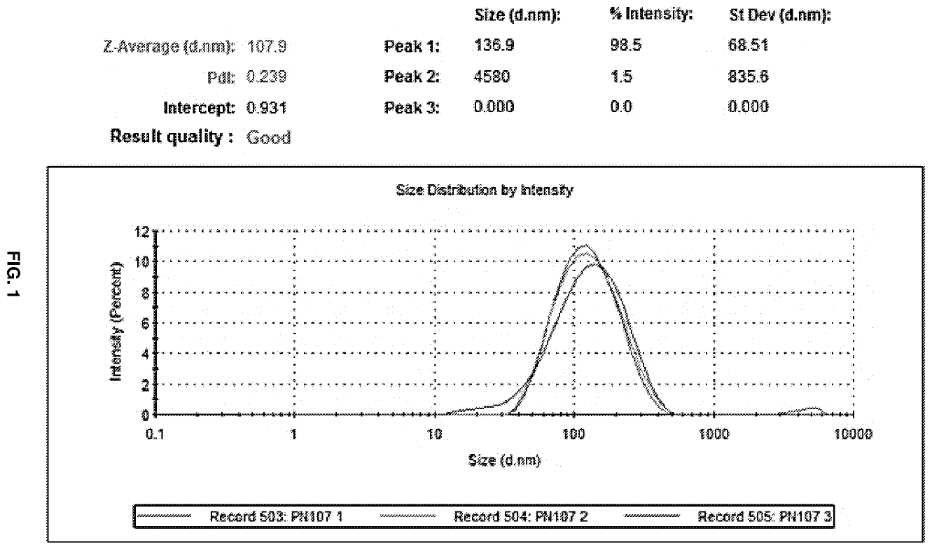

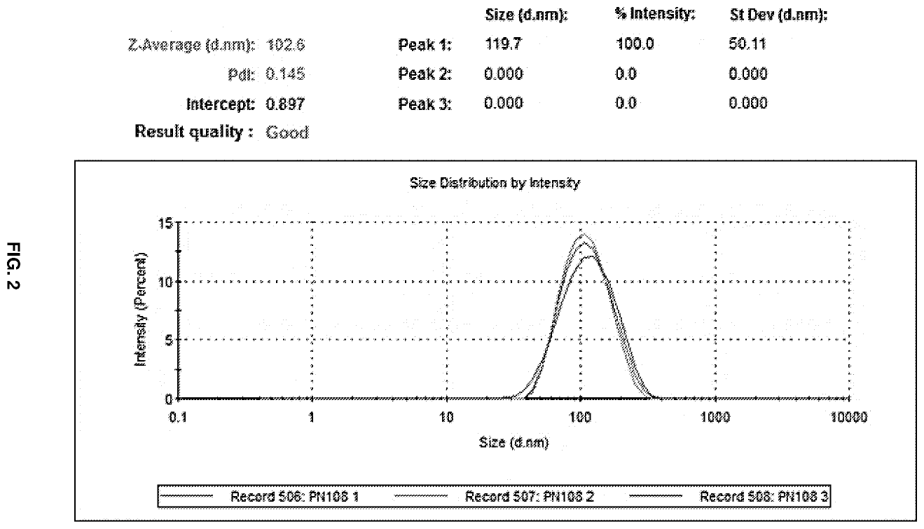

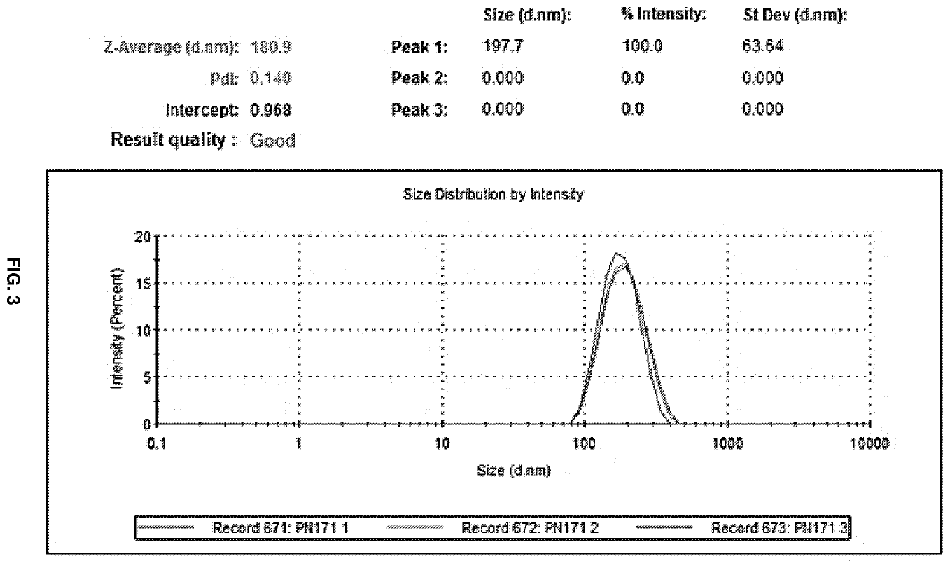

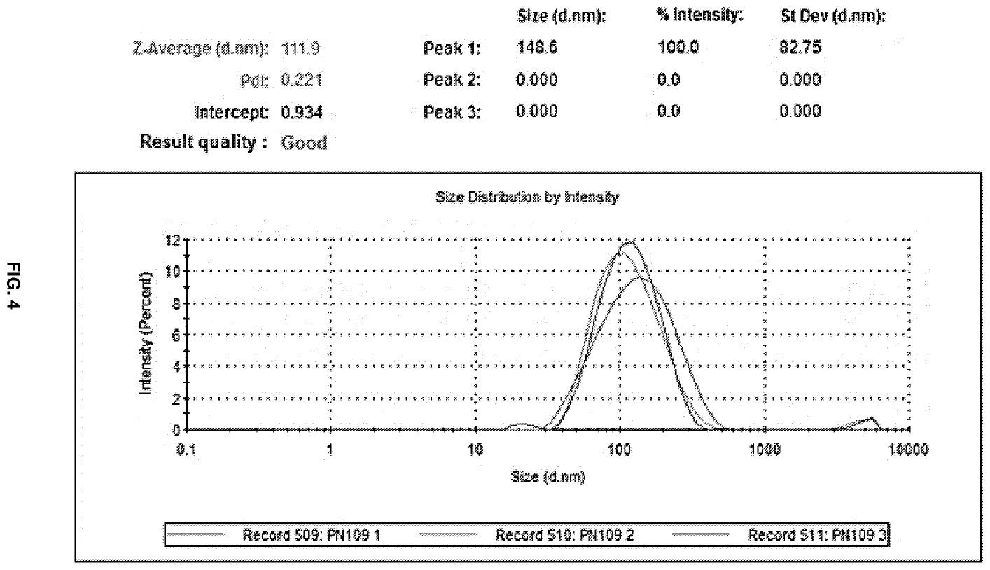

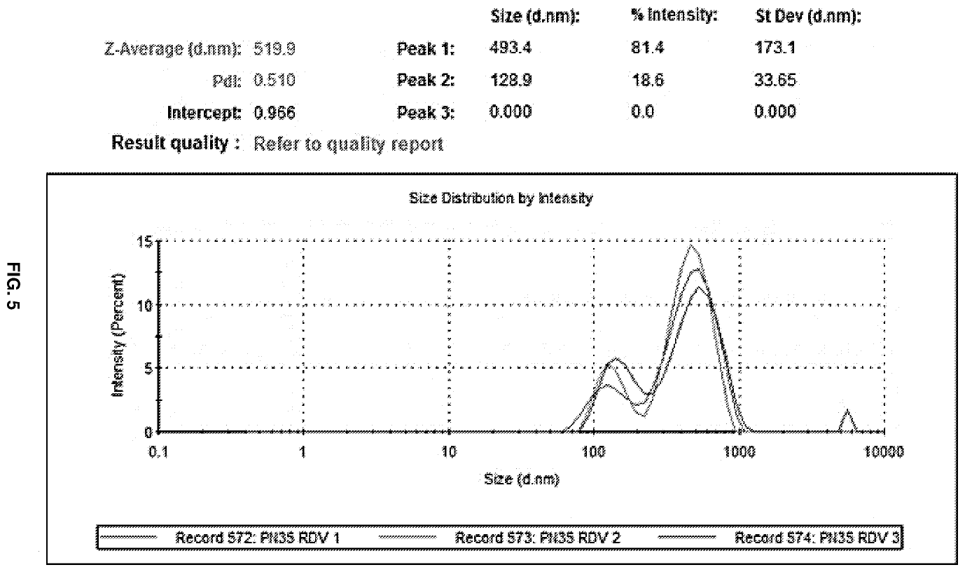

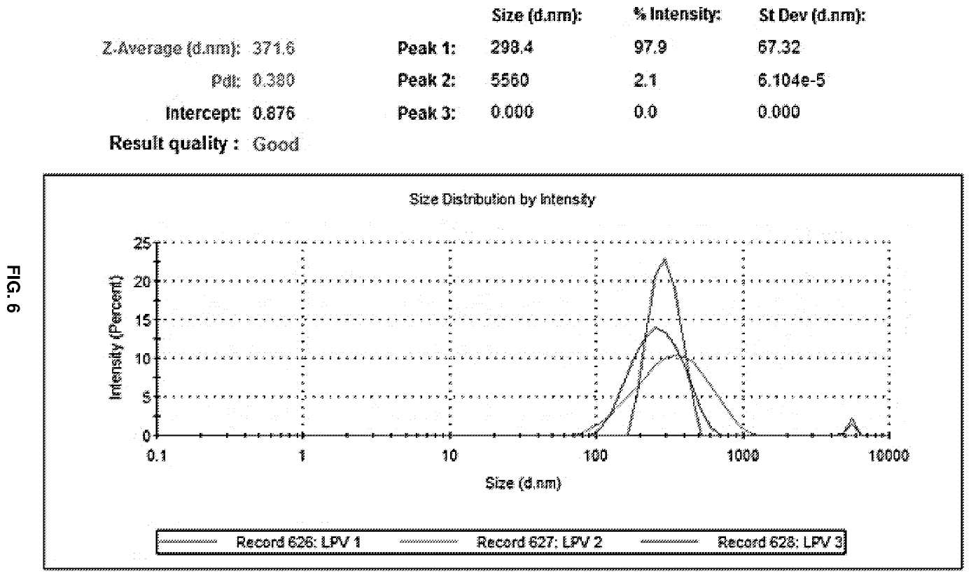

The microemulsion systems (>95% water) incorporating ETB, RDV and LPV drugs were successfully developed using the oil-in-water (O/W) single emulsion via rapid nanoprecipitation technique and the nanoparticle formulations. The measurements of the hydrodynamic size (nanometer, nm) and distribution of both delivery systems were confirmed by DLS. The hydrodynamic sizes and size distributions of both delivery systems encapsulating antiviral drugs were appreciable. The results of the microemulsion systems can be seen in FIG. 1 (ETB), FIG. 2 (RDV), FIG. 3 (LPV) and FIG. 4 (ETB+RDV). The size distributions of the nanoparticles were not as good as the microemulsions, however the powders were redispersable in polar medium (water) and a critical factor was observed. The diffusion rate of nanoparticles with drugs in water was slower and required a few minutes to dissociate. FIG. 5 (RDV) and FIG. 6 (LPV) show the results of nanoparticles encapsulating the antiviral drugs.

The stability of microemulsions and nanoparticle formulations were determined by continuous DLS analysis and the results suggest optimal parameters were achieved for the preparation methods. The size and size distributions were found to be the same after a period of 2 months suggesting good stability and the stability studies are still ongoing.

3.2 Characterization

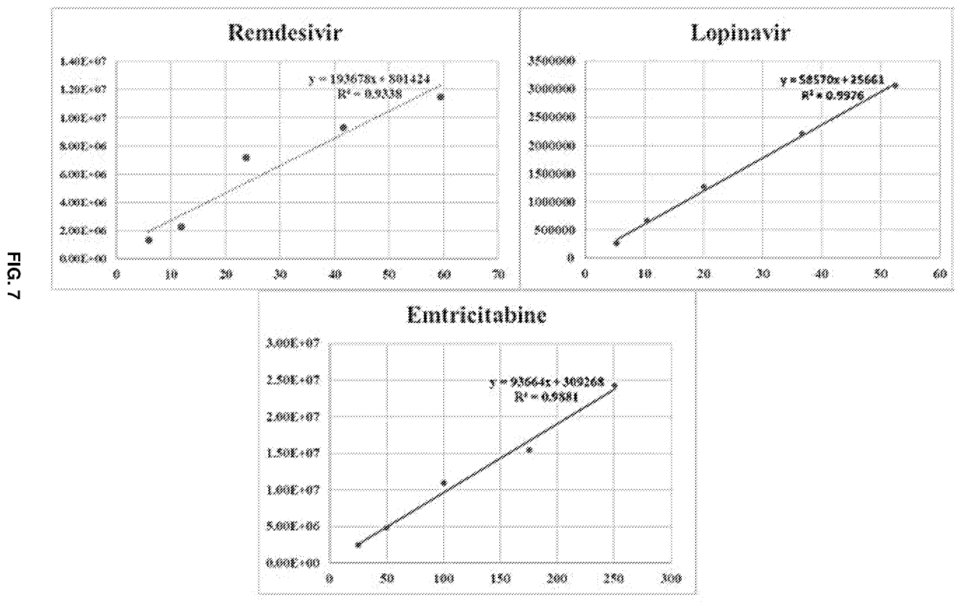

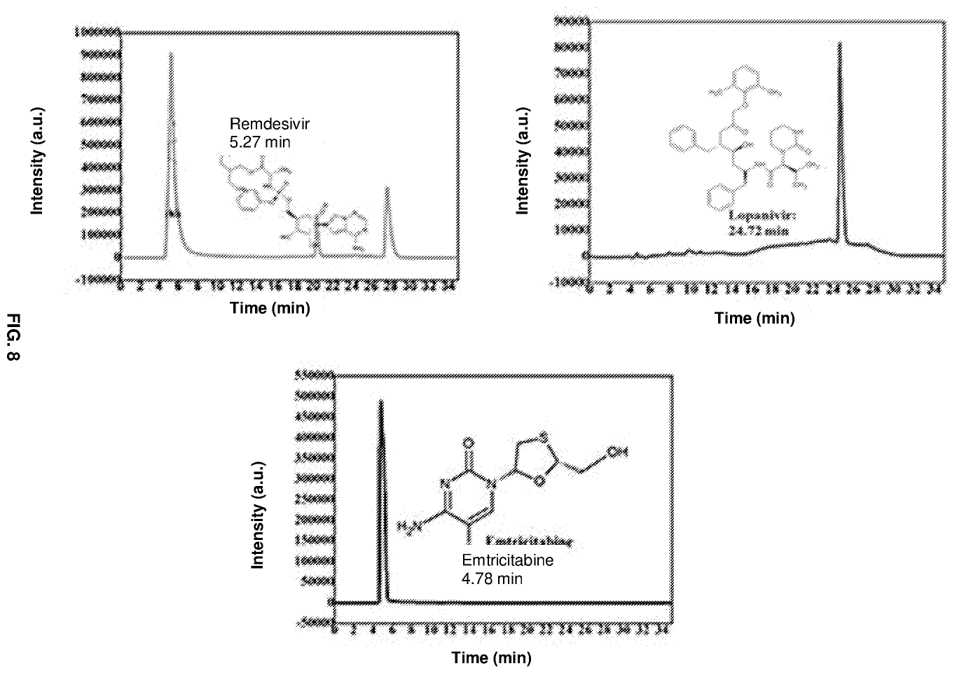

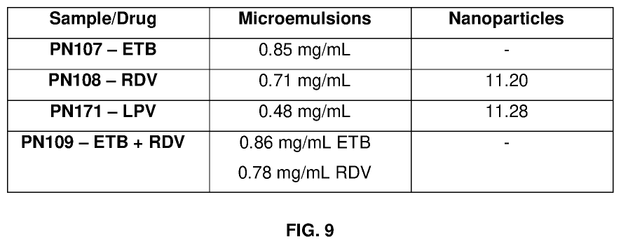

Qualitative and quantitative analysis of the drugs were performed by an HPLC. FIG. 7 below shows the calibration curves of the pure drugs and FIG. 8 shows the retention peaks of drugs in formulations. FIG. 9 shows the achieved drug loadings of the microemulsions and the nanoparticle formulations.

### EXAMPLE 2

### Cannabidiol (Log P 6)—Phytochemical Analgesic Drug

1. Background

The current recommended strategies for preventing infection and the spread of the coronavirus 2019 (COVID-19) have shown little success. The SARS-CoV-2 spike proteins are class 1 viral fusion proteins that mediate infection and have high binding affinity towards the human angiotensin-converting enzyme 2 (hACE2). Pulmonary cells are highly susceptible to infection due to high expression of hACE2 receptors and the innate immune response exaggerates the severity of the disease through its secretion of toxic chemicals (cytokine storm). To mitigate both issues, we employ the use our multifunctional microemulsion drug delivery system incorporating two immunomodulatory drugs, Cannabidiol and Chloroquine. Chloroquine and its derivative, hydroxychloroquine are alkaline molecules that are widely known for their anti-malarial activity since the 1940s. They are primarily absorbed in the gastrointestinal tract, reaching plasma maximum concentrations (Cmax) in less than an hour (±30 min) and usually administered orally. Distribution in cell tissue is rapid followed by entrapment by membrane-enclosed organelles such as endosomes and lysosomes. Their widely proposed and accepted mode of action infections is their lysosomotrophic property. The entrapment by lysosomes results in the alkalization of the organelle which counteracts the normal acidification process necessary for optimal organelle functionality. Furthermore, it has also been shown to have affinity towards allosteric sites that negatively affect normal allosteric regulations resulting in the disruption of membrane bound receptor/protein activity. The potential use CQ was investigated in in-vitro biological models against the SARS-CoV-2 and showed potential of use.

Cannabidiol (CBD) is a naturally occurring chemical compound or phytochemical that is found in cannabis plants. It is one of the 113 cannabinoid compound extracts from cannabis plants and it is the major phytocannabinoid compound which makes up 40% of the total plant extracts. It belongs to the cannabinoid drug class and can be administered through inhalation with bioavailabilities ranging from 11-45% and orally with only 13-19% bioavailability. The extract can be administered in a solution form for oral administration or as an additive in food preparation. It has major medicinal benefits to humans including pain and inflammation relief, anxiety management, seizure control and also has antioxidant properties. The extract is a water insoluble (0.0126 mg/mL), colourless crystalline powder and it is soluble in a various organic solvents. CBD is highly insoluble in water thus impeding absorption and is also subjected to significant first-pass metabolism. Both these properties are major limitations to treatment outcomes and also contribute to its low bioavailability when orally administrated.

2. Methods and Materials

2.1 Materials

The solvents were all purchased from Sigma and include ethanol, acetone, acetonitrile, dimethyl sulfoxide (DMSO), ethyl acetate, dichloromethane (DCM) and oleic acid. Polyvinyl alcohol (PVA) (87-89 hydrolysed/Mw=13000-23000), polyethylene glycol (PEG) (Mw—4000), stearic acid and phosphate buffer saline (PBS—pH 7.4) reagents were all obtained from Sigma Aldrich, South Malvern Zetasizer nano series ZS (DLS) was used for the size and size distribution of the micro-emulsions.

The human epithelial cervical cancer cell line HeLa obtained from the American Type Culture Collection (ATCC, Arlington, Va., USA). Dulbecco's Modified Eagle's Medium (DMEM), fetal calf serum (FCS), antibiotics (penicillin/streptomycin, (pen/strep) and trypsin-EDTA were purchased from Gibco and Pierce (Thermo Fischer Scientific, Johannesburg, South Africa). The FuGENE transfection reagents and Bright-Gloluciferase assay kit were purchased from Promega, USA.

2.2 Methods

2.2.1 Microemulsion Formulation

Cannabidiol (CBD)

CBD (10 to 20 mg) was dissolved in stearic acid and a co-solution of acetone/ethanol, and then 10 to 20 μl of a surfactant with a high HLB value above 10 (Tween 80®) was added to assist in the formation of the oil phase droplets. It is also possible to optionally dissolve PLGA, or alternatively, any biocompatible and biodegradable polymer suitable for use in drug delivery, including polylactic acid, polyglycolic acid, or poly ϵ-caprolactone, in the stearic acid and acetone/ethanol co-solution to further improve stability of the hydrophobic active in the inner matrix of the microemulsion. The continuous polar phase was prepared by mixing equal portions of one buffering solution of phosphate buffer saline (PBS pH 7.4) with two hydrophilic polymers, polyvinyl alcohol (PVA) and polyethylene glycol (i.e. PEG 4000).

To form the liquid microemulsion, the organic phase was rapidly dispensed into an aqueous solution mixture of the continuous polar phase. The organic phase may optionally first be heated to about 40° C., then, when dispensed into the aqueous mixture of hydrophilic polymers, may be moderately stirred using a magnetic hot plate at about 40° C., resulting in a stable microemulsion with reproducible droplet size and size distribution. The system was then stirred under fumehood for 2 hours to evaporate the solvents. A delivery system without the addition of the immunomodulatory drug was also prepared following the exact method of synthesis as described above. The microemulsions were transparent, stable for over 3 months and had a light blue distinct appearance of a phenomenon known as the Tyndall effect.

Chloroquine (CQ)

The internal phase (organic) was prepared by dissolving stearic acid in a co-solution of acetone/ethanol, and then 10 to 20 μl of a surfactant with a high HLB value above 10 (Tween 80®) was added to assist in the formation of the oil phase droplets. It is also possible to optionally dissolve PLGA, or alternatively, any biocompatible and biodegradable polymer suitable for use in drug delivery, including polylactic acid, polyglycolic acid, or poly ϵ-caprolactone, in the stearic acid and acetone/ethanol co-solution to further improve stability of a hydrophobic active, if present, in the inner matrix of the microemulsion. The continuous polar phase was prepared by mixing equal portions of one buffering solution of phosphate buffer saline (PBS pH 7.4) with two hydrophilic polymers, polyvinyl alcohol (PVA) and polyethylene glycol (i.e. PEG 4000). Chloroquine (10-100 mg) was added to and dissolved in the continuous polar phase.

To form the microemulsion, the organic phase was added to the continuous phase while stirring moderately at room temperature (23-25° C.). The spontaneous precipitation resulted in the self-assembly of a thermodynamically stable microemulsion via nucleation. The system was then stirred under fumehood for 2 hours to evaporate the solvents. The microemulsions were transparent, stable for over 3 months and had a light blue distinct appearance of a phenomenon known as the Tyndall effect.

Cannabidiol and Chloroquine (CBD/CQ)

CBD (10 to 20 mg) was dissolved in stearic acid and a co-solution of acetone/ethanol, and then 10 to 20 μl of a surfactant with a high HLB value above 10 (Tween 80®) was added to assist in the formation of the oil phase droplets. It is also possible to optionally dissolve PLGA, or alternatively, any biocompatible and biodegradable polymer suitable for use in drug delivery, including polylactic acid, polyglycolic acid, or poly ϵ-caprolactone, in the stearic acid and acetone/ethanol co-solution to further improve stability of the hydrophobic active in the inner matrix of the microemulsion. The continuous polar phase was prepared by mixing equal portions of one buffering solution of phosphate buffer saline (PBS pH 7.4) with two hydrophilic polymers, polyvinyl alcohol (PVA) and polyethylene glycol (i.e. PEG 4000). Chloroquine (10-100 mg) was added to and dissolved in the continuous polar phase.

To form the microemulsion with both CBD and CQ, the organic phase was added to the continuous phase while stirring moderately at room temperature (23-25° C.). The spontaneous precipitation resulted in the self-assembly of a thermodynamically stable microemulsion via nucleation. The system was then stirred under fumehood for 2 hours to evaporate the solvents. The organic phase may optionally first be heated to about 40° C., then, when dispensed into the aqueous mixture of hydrophilic polymers, may be moderately stirred using a magnetic hot plate at about 40° C., resulting in a stable microemulsion with reproducible droplet size and size distribution. The microemulsions were transparent, stable for over 4 months and had a light blue distinct appearance of a phenomenon known as the Tyndall effect.

2.2.2 Physicochemical Characterization

2.2.2.1 Hydrodynamic Size, Size Distribution and Stability

The hydrodynamic size, size distribution and stability of the delivery system was determined by a Dynamic Light Scattering (DLS) technique using the Malvern Zetasizer Nano series ZS. The DLS instrument measures the Brownion motion, the random movement (fluctuation) of submicrom particles in a solution to determine the hydrodynamic size. Briefly, a laser beam is used to illuminate the sample solution, the incident laser beam gets scattered in all direction and the intensity measured by a detector. To elaborate, continuous data correlation of the speed and the count rate in kilocounts per second (Kcps) of particle diffusion in solution are the key parameters for size determination. The smaller particles in solution diffuse faster than the larger particles. Stability of submicron particles can also be determined by DLS over time through continuous sample analysis. Samples for analysis were prepared in deionized water, diluted 300 to 400 times and a disposable zetasizer cuvette was used for the analysis.

2.2.2.2 Qualitative and Quantitative Analysis

The analytical methods used were developed by following the guidelines from the U.S. Department of Health and Human Services Food and Drug Administration on Analytical Procedures and Methods Validation for Drugs and Biologics, Guidance for Industry published in 2015 and European Pharmacopoeia (EP10.0). The HPLC separation was carried out using a phenomenex LUNA C₁₈ column (150×4.6 mm id; 5 micron particle size) and a mobile phase composed of methanol (A) and 0.1% aqueous triethylamine, pH 3.2 adjusted with phosphoric acid (B), at a flow rate 0.4 mL/min. The gradient elution programme was 10% A from 0-1.9 min, 10-40% A from 1.9-2.0 min and 40% A from 2.0-3.3 min. For column re-equilibration, 10% A was maintained from 3.3-5.00 min. UV detection was performed at 220-260 nm and the injection volume was 20 μl.

A simple, rapid, and selective reversed-phase high-performance liquid-chromatography (HPLC-UV) method was developed for the determination of Cannabidiol and Chloroquine in the microemulsion formulations. Calibration curves were prepared by the analysis of blank delivery system samples spiked with various concentrations of working solutions of the drug substances. The samples were then submitted to the processes such as sonication, chromatographic separation, and UV detection described above. Calibration curves were obtained by linear least-squares regression analysis plotting of peak areas versus the concentrations. The calibration curve equation is y=ax+b, where y represents the peak areas and x represents the concentrations of the drug substances. The limit of detection (LOD) was determined as the lowest concentration giving a signal to noise ratio (S/N) of 3 for all of the drug substances. Limit of quantification (LOQ), the lowest amount of analyte that can be quantified with acceptable precision and accuracy, was determined as S/N of 10.

Stock solutions of the drugs were prepared in methanol/water (50:50). Prior to measurements, stock solutions were diluted with methanol-water (50:50, v/v) so as to prepare the working standard solutions of 100 μg/mL and 1 μg/mL. Various dilutions were made to prepare working solutions. HPLC analysis was carried out with 20 μL aliquots of various concentrations of the working solutions.

2.2.2.3 Biological Testing

Pseudovirus Neutralization Assay

The inhibition activity of both the CBD and CQ microemulsion delivery systems were tested in a TZM-bl neutralization assay. The TZM-bl neutralization assay mimics the inhibition of free viral particles infection of cells. Briefly, the TZM-bl neutralization assay was performed by preparing a dilution series of the inhibitors in 100 μL of the growth medium (DMEM) with 10% Fetal Bovin Serum (FBS) in a 96-well plate in duplicate. This was followed by the addition of 100 TCID₅₀ of pseudovirus in 50 μL of growth medium and incubated for one hour at 37° C. Then 100 μL of TZM-bl cells at a concentration of 1×10⁵ cells/mL containing 37.5 μg/mL of DEAE-dextran will be added to each well and cultured at 37° C. for 48 h. Infection will be evaluated by measuring the activity of the firefly luciferase.

Titers were calculated as the inhibitory dilution that causes 50% reduction (ID₅₀) of relative light unit (RLU) compared to the virus control (wells with no inhibitor) after the subtraction of the background (wells without both the virus and the inhibitor). The luciferase assay was performed with the Bright-Gloluciferase assay kit (Promega, USA) according to the manufacturer's instructions and luciferase activity has been expressed in terms of relative luciferase units (RLUs). The assay described above will be adapted to test for the inhibition of SARS-CoV-2 pseudovirus infection employing the use of 293-T cells instead of TZM-bl cells.

3. Results

3.1 Delivery System

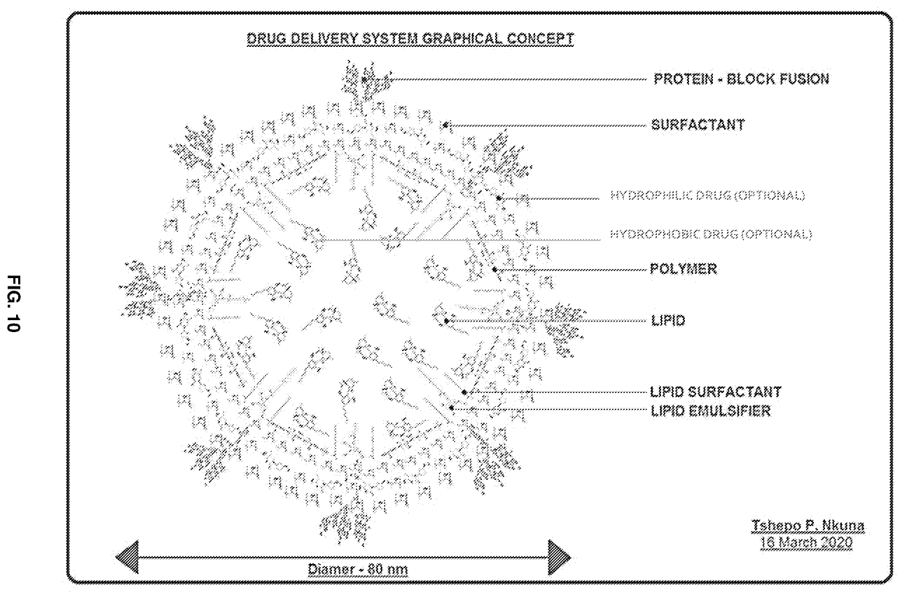

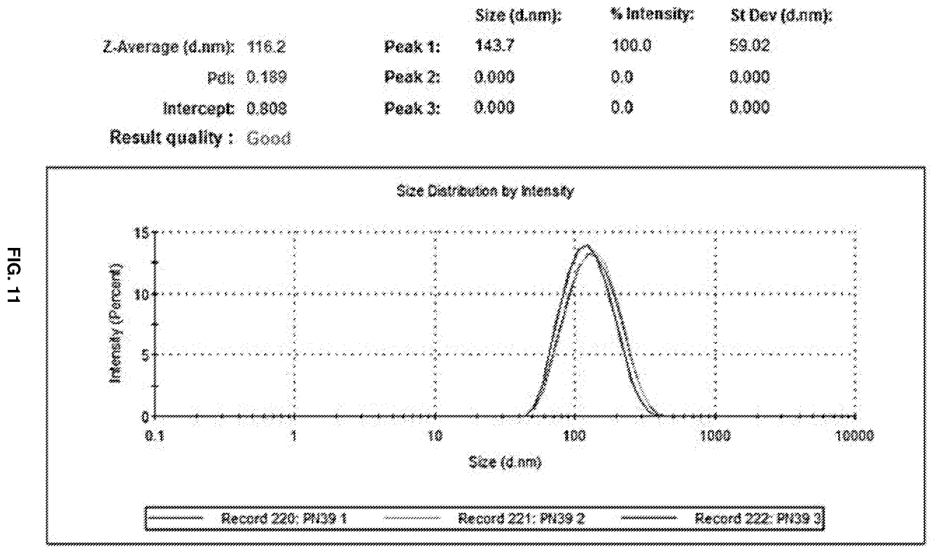

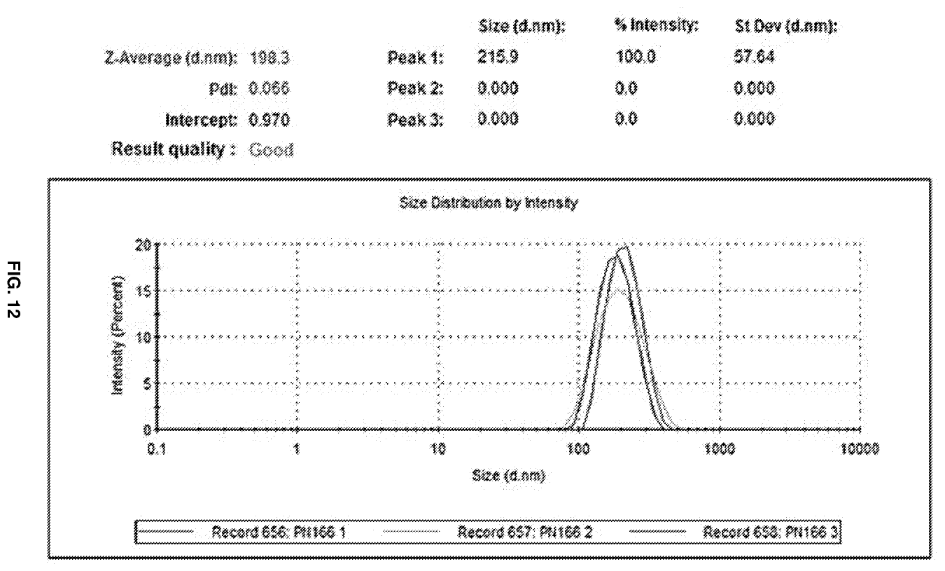

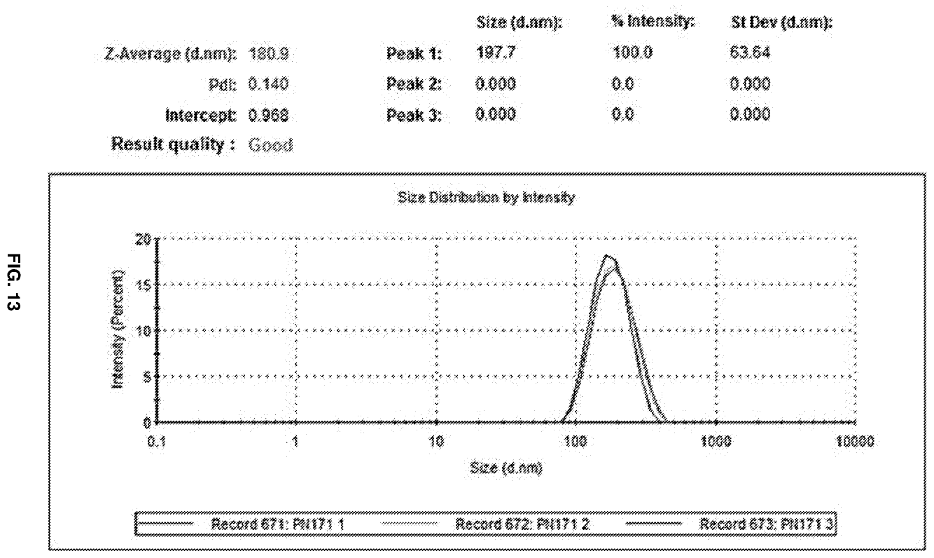

The microemulsion systems (>95% water) incorporating CBD, CQ and the combination of the two drugs were successfully developed using the oil-in-water (O/W) single emulsion via rapid nanoprecipitation technique. The measurements of the hydrodynamic size (nanometer, nm) and distribution of the microemulsion was confirmed by DLS and FIG. 10 below depicts a graphical representation of a nanodroplet (internal phase) dispersed homogeneously throughout the continuous phase. The hydrodynamic sizes and size distributions of microemulsion systems with immunomodulatory drugs were appreciable and the results can be seen in FIG. 11 (CBD), FIG. 12 (CQ) and FIG. 13 (CBD+CQ).

The stability of microemulsions was determined by continuous DLS analysis and the results suggest optimal parameters were achieved for the preparation methods. The size and size distributions were found to be the same after a period of 4 months suggesting good stability.

3.2 Characterization

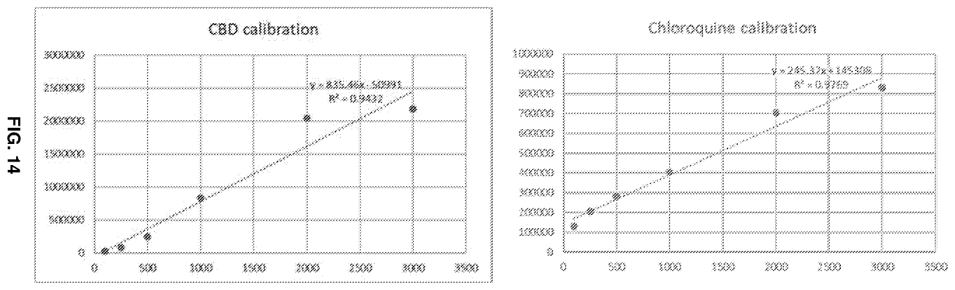

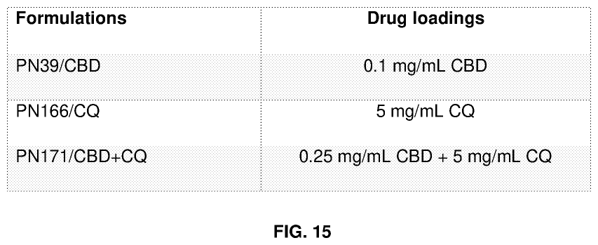

Qualitative and quantitative analysis of the drugs were performed by an HPLC. FIG. 14 below shows the calibration curves of the pure drugs and FIG. 15 shows the achieved drug loadings of the microemulsion formulations.

3.3 Pseudovirus Neutralization Assay

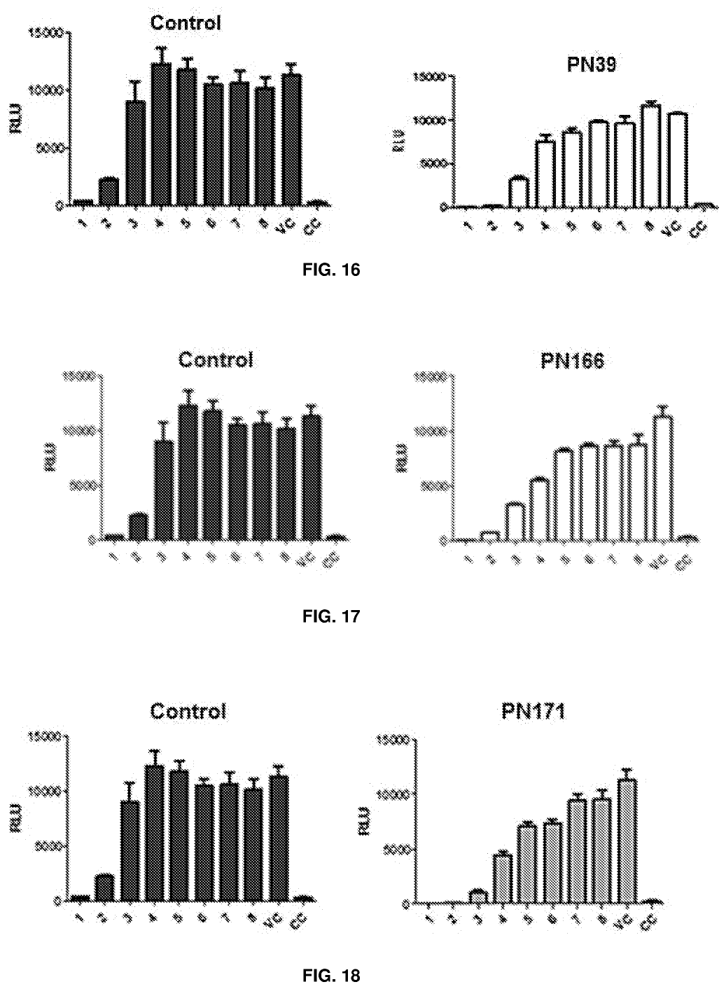

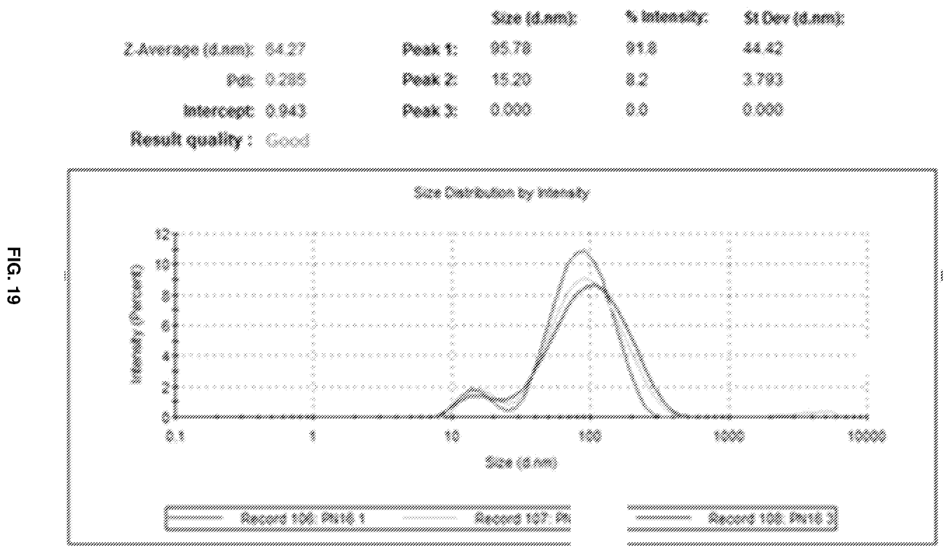

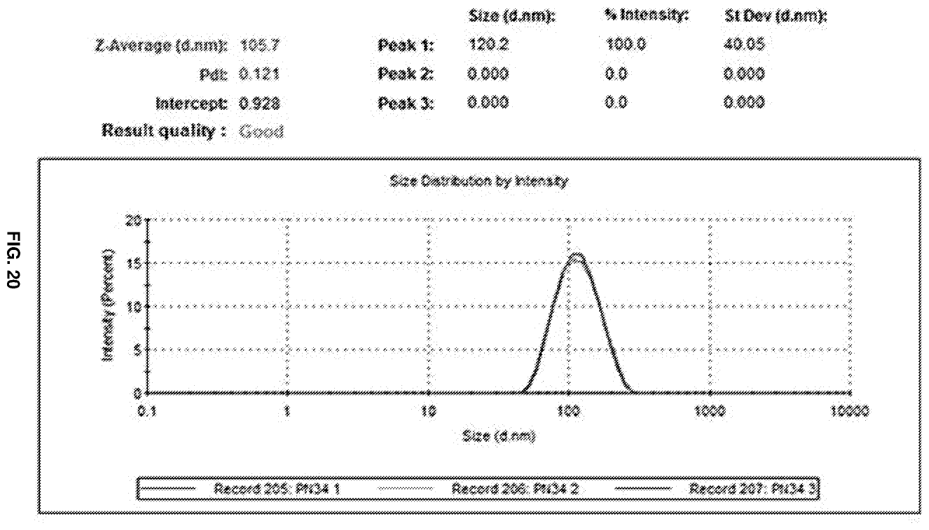

The antiviral activity of CBD (FIG. 16), CQ (FIG. 17) and the combination (FIG. 18) was demonstrated using the TZM-bl neutralization assay and successful inhibition of the pseudovirus from infecting the cells was observed.

### EXAMPLE 3

### Method for Preparation of a Novel Microemulsion Delivery System Functionalized with Antiviral Cyanovirin-N and Griffithsin for Prevention of COVID-19

4. Background

Cellular entry by the SARS-CoV-2 is a two-step mechanism mediated by fusion of the receptor-binding domain (RBD), the spike (S) glycoprotein to the human angiotensin-converting enzyme 2 (hACE2). The domain has high binding affinity towards the hACE2 and protease cleavage is necessary for activation by cell surface proteases such as TMPRSS2 and lysosomal proteases cathepsins. The RBD has two subunits, the S1 receptor-binding subunit responsible for attachment and the S2 membrane fusion subunit for cell entry through endocytosis. Post viral attachment, the S1 subunit dissociates allowing a major structural configuration of the S2 subunit resulting in endositic uptake for infection. The SARS-CoV-2 spike proteins are class 1 viral fusion proteins that mediate both the attachment and cellular entry of the virus.

Cyanovirin-N and griffithsin are broad spectrum antiviral proteins that inhibit the function of class 1 fusion proteins. The virucidal effects have been shown against multiple viruses including HPV, HIV and a few enteric viruses. These viruses use their surface hemagglutinin (HE) protein, a class 1 fusion protein for attachment to target cells followed by an endocytic uptake resulting in infection. Cyanovirin-N and griffithsin have high binding affinity towards these surface glycoproteins of viruses and through binding the proteins envelopes the virus HE inhibiting their fusion to the target cells. The SARS-CoV-2 also has this class 1 fusion protein on its surface and is the main target for inhibiting infection.

5. Methods and Materials

a. Materials

Cyanovirin-N and griffithsin were supplied by the NextGen Health cluster of the CSIR. Solvents were all purchased from Sigma and include ethanol, acetone, acetonitrile, dimethyl sulfoxide (DMSO), ethyl acetate, dichloromethane (DCM) and oleic acid. Polyvinyl alcohol (PVA) (87-89 hydrolysed/Mw=13000-23000), polyethylene glycol (PEG) (Mw—4000), stearic acid and phosphate buffer saline (PBS—pH 7.4) reagents were all obtained from Sigma Aldrich, South Africa. Malvern Zetasizer nano series ZS (DLS) was used to determine the hydrodynamic size, size distribution and the stabily of the microemulsions and a Shimadzu SIL-20AXR/20ACRXR prominence High Pressure Liquid Chromatography (HPLC) for qualitative analysis.

The human epithelial cervical cancer cell line HeLa obtained from the American Type Culture Collection (ATCC, Arlington, Va., USA). Dulbecco's Modified Eagle's Medium (DMEM), fetal calf serum (FCS), antibiotics (penicillin/streptomycin, (pen/strep) and trypsin-EDTA were purchased from Gibco and Pierce (Thermo Fischer Scientific, Johannesburg, South Africa). The FuGENE transfection reagents and Bright-Gloluciferase assay kit were purchased from Promega, USA.

2.2 Methods

The design and development of the delivery system considered a variety of lipids, polymers, solvents and surfactants suitable for the application to achieve desired physicochemical properties. Also, the selection of raw materials considered the route of administration, the target sites and the most critical consideration was to select materials that safe for human consumption and approved by international regulatory bodies such as the South African Health Practitioner Regulatory Authority (SAHPRA) and the Food and Drug Administration (FDA). The polymer and lipid used are biodegradable and biocompatible, the solvents and volumes used of are within the recommended and allowable limits, and critical factors such as concentrations and ratios were investigated in order to achieve an optimal delivery system.

2.2.1 Synthesis of the Delivery System

Briefly, the microemulsion system functionalized with Cyanovirin-N/Griffithsin was prepared as follows: The organic phase (internal) was prepared by dissolving stearic acid and PLGA (1:5 ratio) in a co-solution of acetone/ethanol followed by the addition of 10 to 20 μl of a surfactant with a high HLB value above 10 (Tween 80®). The continuous polar phase was prepared by mixing equal portions of one buffering solution of phosphate buffer saline (PBS pH 7.4) with two hydrophilic polymers, polyvinyl alcohol (PVA) and polyethylene glycol (i.e. PEG 4000). The stock solution of the antiviral lectin was prepared by dissolving 0.1-1 mg in a PBS (pH 7.4) solution and 10-100 μL was added to the continuous phase.

To form the microemulsion with the antiviral lectin, the organic phase was added to the continuous phase while stirring moderately at room temperature (23-25° C.). The spontaneous precipitation of the SA/PLGA resulted in the self-assembly of a thermodynamically stable microemulsion via nucleation. The system was then stirred under fumehood for 2 hours to evaporate the solvents. A delivery system without the addition of lectins was also prepared following the exact method of synthesis as described above. The microemulsions were transparent, stable for over 3 months and had a light blue distinct appearance of a phenomenon known as the Tyndall effect.

2.2.2 Physicochemical Characterization

a) Hydrodynamic Size, Size Distribution and Stability

The hydrodynamic size, size distribution and stability of the delivery system was determined by a Dynamic Light Scattering (DLS) technique using the Malvern Zetasizer Nano series ZS. The DLS instrument measures the Brownion motion, the random movement (fluctuation) of submicrom particles in a solution to determine the hydrodynamic size. Briefly, a laser beam is used to illuminate the sample solution, the incident laser beam gets scattered in all direction and the intensity measured by a detector. To elaborate, continuous data correlation of the speed and the count rate in kilocounts per second (Kcps) of particle diffusion in solution are the key parameters for size determination. The smaller particles in solution diffuse faster than the larger particles. Stability of submicron particles can also be determined by DLS over time through continuous sample analysis. Samples for analysis were prepared in deionized water, diluted 300 to 400 times and a disposable zetasizer cuvette was used for the analysis.

b) Qualitative and Quantitative Analysis

The analytical methods used were developed by following the guidelines from the U.S. Department of Health and Human Services Food and Drug Administration on Analytical Procedures and Methods Validation for Drugs and Biologics, Guidance for Industry published in 2015 and European Pharmacopoeia (EP10.0). The HPLC separation was carried out using a phenomenex LUNA C₁₈ column (150×4.6 mm id; 5 micron particle size) and a mobile phase composed of methanol (A) and 0.1% aqueous triethylamine, pH 3.2 adjusted with phosphoric acid (B), at a flow rate 0.4 mL/min. The gradient elution programme was 10% A from 0-1.9 min, 10-40% A from 1.9-2.0 min and 40% A from 2.0-3.3 min. For column re-equilibration, 10% A was maintained from 3.3-5.00 min. UV detection was performed at 220-260 nm and the injection volume was 20 μl.

A simple, rapid, and selective reversed-phase high-performance liquid-chromatography (HPLC-UV) method was developed for the determination of Cyanovirin-N and Griffithsin in the microemulsion formulations. Calibration curves were prepared by the analysis of blank delivery system samples spiked with various concentrations of working solutions of the drug substances. The samples were then submitted to the processes such as sonication, chromatographic separation, and UV detection described above. Calibration curves were obtained by linear least-squares regression analysis plotting of peak areas versus the concentrations. The calibration curve equation is y=ax+b, where y represents the peak areas and x represents the concentrations of the drug substances. The limit of detection (LOD) was determined as the lowest concentration giving a signal to noise ratio (S/N) of 3 for all of the drug substances. Limit of quantification (LOQ), the lowest amount of analyte that can be quantified with acceptable precision and accuracy, was determined as S/N of 10.

Stock solutions of the antiviral lectins were prepared in methanol/water (50:50). Prior to measurements, stock solutions were diluted with methanol-water (50:50, v/v) so as to prepare the working standard solutions of 100 μg/mL and 1 μg/mL. Various dilutions were made to prepare working solutions. HPLC analysis was carried out with 20 μL aliquots of various concentrations of the working solutions.

2.2.3 Biological Testing

c) Pseudovirus Neutralization Assay

The inhibition activity of both the Cyanovirin-N and Griffithsin microemulsion delivery systems were tested in a TZM-bl neutralization assay. The TZM-bl neutralization assay mimics the inhibition of free viral particles infection of cells. Briefly, the TZM-bl neutralization assay was performed by preparing a dilution series of the inhibitors in 100 μL of the growth medium (DMEM) with 10% Fetal Bovin Serum (FBS) in a 96-well plate in duplicate. This was followed by the addition of 100 TCID₅₀ of pseudovirus in 50 μL of growth medium and incubated for one hour at 37° C. Then 100 μL of TZM-bl cells at a concentration of 1×10⁵ cells/mL containing 37.5 μg/mL of DEAE-dextran will be added to each well and cultured at 37° C. for 48 h. Infection will be evaluated by measuring the activity of the firefly luciferase.

Titers were calculated as the inhibitory dilution that causes 50% reduction (ID₅₀) of relative light unit (RLU) compared to the virus control (wells with no inhibitor) after the subtraction of the background (wells without both the virus and the inhibitor). The luciferase assay was performed with the Bright-Gloluciferase assay kit (Promega, USA) according to the manufacturer's instructions and luciferase activity has been expressed in terms of relative luciferase units (RLUs). The assay described above will be adapted to test for the inhibition of SARS-CoV-2 pseudovirus infection employing the use of 293-T cells instead of TZM-bl cells.

6. Results

a. Delivery System

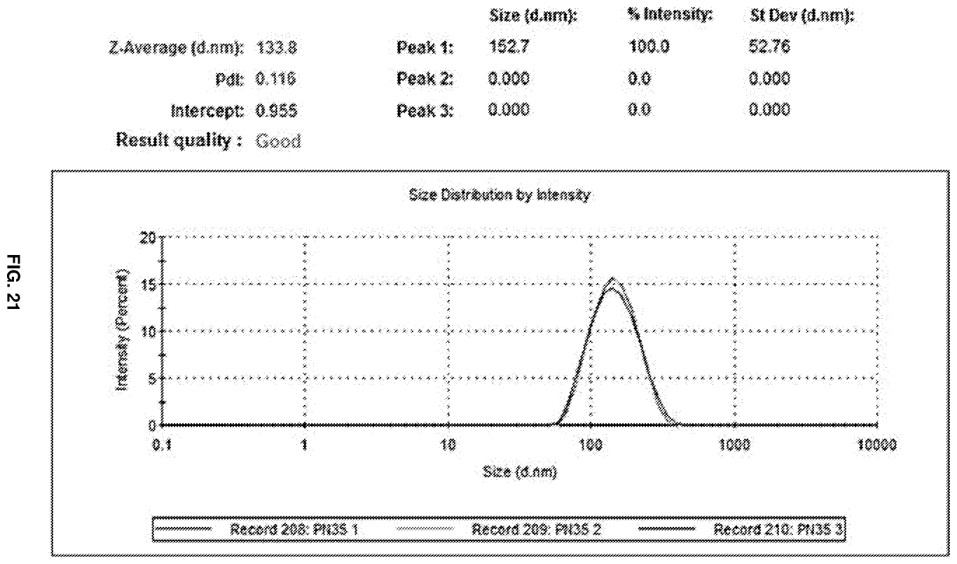

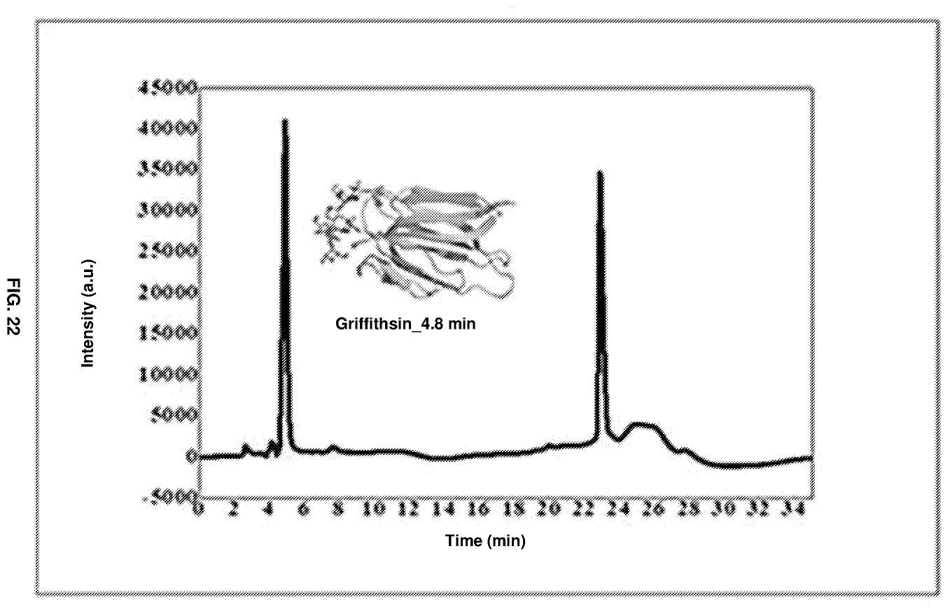

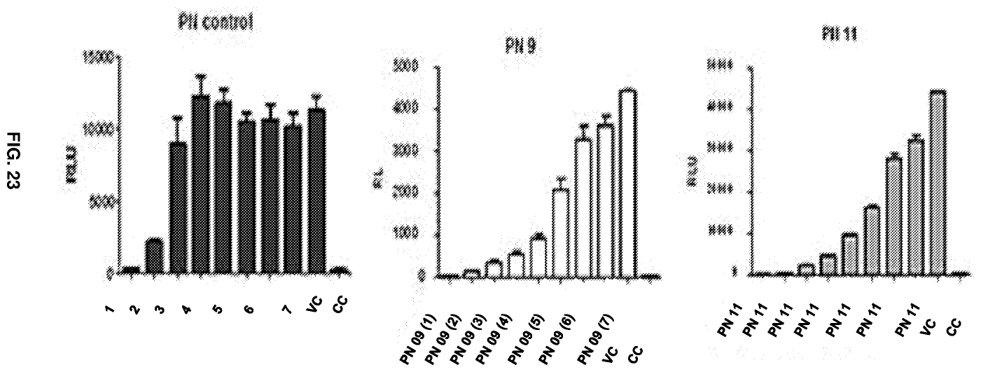

The microemulsion systems (>95% water) functionalized with either CVN or GFTS were successfully developed using the oil-in-water (O/W) single emulsion via rapid nanoprecipitation technique. The microemulsion system without the addition of the antiviral lectins had narrow size distributions with an average size of 83.19 nm in diameter (FIG. 19). As can be seen in FIGS. 20 (CVN) and 21 (GFTS), the microemulsion systems incorporating the lectins increased in size by at least 22.51 nm for CVN and 50.61 nm for GTS.

The stability of microemulsions was determined by continuous analysis and it is to be appreciated that an increase of the count rate of nanodroplets was observed during the nucleation process of forming the microemulsion. This suggests optimal parameters were achieved for the preparation method. The size and size distributions were found to be the same after a period of 3 months suggesting good stability.

b. Characterization

To confirm the lectins integrity in the system, a qualitative analysis by an HPLC was conducted and FIG. 22 below shows a perfect retention peak of an intact antiviral lectin post formulation.

c. Pseudovirus Neutralization Assay

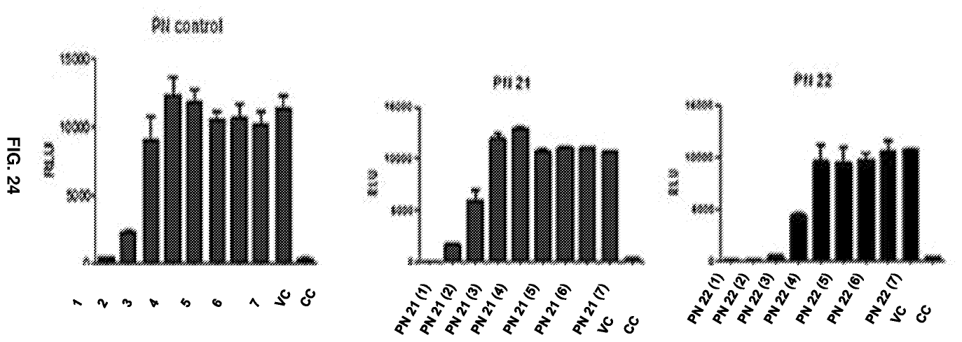

The antiviral activity of the lectins was demonstrated using the TZM-bl neutralization assay, FIG. 23 (CVN) and FIG. 24 (GFTS) show successful inhibition of the pseudovirus from infecting the cells.

1. A polymer-lipid microemulsion drug delivery system for the treatment or inhibition of viral Acute Respiratory Distress Syndromes (ARDS) comprising or consisting of:
i. an inner microemulsion matrix comprised or consisting of at least one fatty acid dissolved in a polar aprotic solvent, and a surfactant;
ii. an outer shell comprising or consisting of one or more hydrophilic polymers; and
iii. one or more drug(s) selected from the group consisting of:
a. antiviral drug(s);
b. immunomodulatory compound(s); and
c. antiviral lectin(s),
wherein where the one or more drug(s) is a hydrophobic drug, the drug is comprised in the inner microemulsion matrix, and wherein the drug is a hydrophilic drug, the antiviral drug is comprised in the outer shell.

2. The drug delivery system according to claim 1, wherein the one or more antiviral drug(s) are selected from hydrophobic antiviral drugs Remdesivir and Lopinavir, and a hydrophilic antiviral drug Emtricitabine.

3. The drug delivery system according to either claim 1 or claim 2, wherein the one or more immunomodulatory compound(s) are selected from hydrophobic cannabidiol (CBD) and hydrophilic chloroquine or chloroquine diphosphate.

4. The drug delivery system according to any one of claims 1 to 3, wherein the one or more antiviral lectin(s) are selected from hydrophilic antiviral lectins griffithsin (GRFT), cyanovirin-N (CV-N), and scytovirin (SVN).

5. The drug delivery system according to claim 4, wherein the antiviral lectins are GRFT and CV-N.

6. The drug delivery system according to any one of claims 1 to 5, wherein the outer shell comprises an aqueous solution of an aqueous mixture of hydrophilic polymers including polyvinyl alcohol (PVA) and polyethylene glycol (PEG), including PEG 4000.

7. The drug delivery system according to any one of claims 1 to 5, wherein the inner microemulsion matrix further comprises at least one organic carboxylic acid, including any one or more of acetic acid, lactic acid, citric acid, or phosphoric acid.

8. The drug delivery system according to claim 7, wherein the organic carboxylic acid is acetic acid.

9. The drug delivery system according to any one of claims 1 to 8, wherein the inner microemulsion matrix comprises at least one copolymer, poly(lactic-co-glycolic acid) or PLGA, or alternatively, any biocompatible and biodegradable polymer suitable for use in active compound or drug delivery, including polylactic acid, polyglycolic acid, or poly ϵ-caprolactone.

10. The drug delivery system according to any one of claims 1 to 9, wherein the at least one fatty acid comprises or consists of any one or more of stearic acid, palmitic acid and lauric acid.

11. The drug delivery system according to claim 10, wherein the fatty acid is stearic acid.

12. The drug delivery system according to any one of claims 1 to 11, wherein the polar aprotic solvent comprises of either ethanol or acetone, or is a blend of ethanol and acetone.

13. The drug delivery system according to claim 10, wherein the polar aprotic solvent is acetone.

14. The drug delivery system according to any one of claims 1 to 13, wherein the surfactant comprises any surfactant having a Hydrophile-Lipophile Balance (HLB) value of greater than 10.

15. The drug delivery system according to claim 14, wherein the surfactant is polysorbate 80.

16. The drug delivery system according to any one of claims 1 to 15, which is isotropic and translucent, having a droplet size of the dispersed phase which is below about 150 nm.

17. The drug delivery system according to any one of claims 1 to 16, wherein the viral ARDS is selected from influenza or SARS-CoV, including SARS-CoV-2 and MERS-CoV.

18. The drug delivery system according to claim 17, wherein the viral ARDS is SARS-CoV-2.

19. A process for producing a polymer-lipid microemulsion drug delivery system comprising one or more drug(s) selected from the group consisting of antiviral drug(s); immunomodulatory compound(s); and antiviral lectin(s), comprising or consisting essentially of the steps of:
A.I. mixing at least one hydrophobic drug, a fatty acid dissolved in a polar aprotic solvent, and a surfactant to form an organic phase;
A.II. optionally heating the organic phase;
A.III. dispensing the organic phase into an aqueous mixture comprising at least one hydrophilic polymer to form a microemulsion; and
A.IV. stabilising the microemulsion in a phosphate buffer at about 0° C. to form the polymer-lipid microemulsion, or
B.I. mixing a fatty acid dissolved in a polar aprotic solvent, and a surfactant to form an organic phase;
B.II. optionally heating the organic phase;
B.III. dispensing the organic phase into an aqueous mixture comprising at least one hydrophilic polymer and at least one hydrophilic drug to form a microemulsion; and
B.IV. stabilising the microemulsion in a phosphate buffer at about 0° C. to form the polymer-lipid microemulsion, or
C.I. mixing at least one hydrophobic drug, a fatty acid dissolved in a polar aprotic solvent, and a surfactant to form an organic phase;
C.II. optionally heating the organic phase;
C.III. dispensing the organic phase into an aqueous mixture comprising at least one hydrophilic polymer and at least one hydrophilic drug to form a microemulsion; and
C.IV. stabilising the microemulsion in a phosphate buffer at about 0° C. to about 10° C. form the polymer-lipid microemulsion.

20. The process according to claim 19, wherein the one or more antiviral drug(s) are selected from the group consisting of hydrophobic antiviral drugs Remdesivir and Lopinavir, and hydrophilic antiviral drug Emtricitabine.

21. The process according to either claim 19 or 20, wherein the hydrophobic immunomodulatory compound is cannabidiol (CBD) and the hydrophilic immunomodulatory compound is selected from the group consisting of chloroquine and chloroquine diphosphate.

22. The process according to any one of claims 19 to 21, wherein the one or more antiviral lectin(s) are selected from the group consisting of hydrophilic antiviral lectins griffithsin (GRFT), cyanovirin-N (CV-N), and scytovirin (SVN).

23. The process according to claim 22, wherein the antiviral lectins are GRFT and CV-N.

24. The process according to any one of claims 19 to 23, wherein the polymer-lipid microemulsion delivery system is a liquid and is nebulised for delivery by inhalation, including for pulmonary delivery.

25. The process according to any one of claims 19 to 23, wherein the process optionally further comprises a final step of drying the stabilised polymer-lipid microemulsion to produce a free flowing polymer-lipid microemulsion powder either by freeze drying or by spray drying.

26. The process according to claim 25, wherein the free flowing polymer-lipid microemulsion delivery system is formulated for oral or intravenous delivery.

27. The process according to any one of claims 19 to 26, further comprising mixing an organic carboxylic acid with the organic phase.

28. The process according to any one of claims 19 to 26, further comprising dissolving at least one biocompatible and biodegradable polymer or copolymer suitable for use in active compound delivery, including poly(lactic-co -glycolic acid) or PLGA, or polylactic acid, polyglycolic acid, or poly ϵ-caprolactone, into the polar aprotic solvent with the fatty acid to form the organic phase.

29. The process according to any one of claims 19 to 28, wherein the least one fatty acid comprises or consists of any one or more of stearic acid, palmitic acid and lauric acid.

30. The process according to claim 29, wherein the fatty acid is stearic acid.

31. The process according to any one of claims 19 to 30, wherein the polar aprotic solvent comprises either ethanol or acetone, or is a blend of ethanol and acetone.

32. The process according to claim 31, wherein the polar aprotic solvent is acetone.

33. The process according to claim 27, wherein the organic carboxylic acid includes any one or more of those approved for human consumption comprising acetic acid, lactic acid, citric acid, or phosphoric acid.

34. The process according to claim 27 or 33, wherein the organic carboxylic acid is acetic acid.

35. The process according to any one of claims 19 to 34, wherein the surfactant comprises any surfactant having a Hydrophile-Lipophile Balance (HLB) value of greater than 10.

36. The process according to claim 35, wherein the surfactant is polysorbate 80.

37. The process according to any one of claims 19 to 36, comprising or consisting of the following steps:
A.a) dissolving at least one fatty acid in a polar aprotic solvent to form a fatty acid solution;
A.b) dissolving at one or more hydrophobic drug(s) in the fatty acid solution;
A.c) adding drop-wise, a surfactant to form an organic phase;
A.d) optionally heating the organic phase;
A.e) dispensing the organic phase into an aqueous mixture comprising at least one hydrophilic polymer, and optionally one or more hydrophilic drug(s) while stirring to form a microemulsion; and
A.f) stabilising the polymer-lipid microemulsion by adding a phosphate buffer at 0° C. while stirring, or
B.a) dissolving at least one fatty acid in a polar aprotic solvent to form a fatty acid solution;
B.b) optionally dissolving one or more hydrophobic drug(s) in the fatty acid solution;
B.c) adding drop-wise, a surfactant to form an organic phase;
B.d) optionally heating the organic phase;
B.e) dispensing the organic phase into an aqueous mixture comprising at least one hydrophilic polymer, and one or more hydrophilic drug(s) while stirring to form a microemulsion; and
B.f) stabilising the polymer-lipid microemulsion by adding a phosphate buffer at 0° C. while stirring, or
C.a) dissolving at least one fatty acid in a polar aprotic solvent to form a fatty acid solution;
C.b) dissolving one or more hydrophobic drug(s) in the fatty acid solution;
C.c) adding drop-wise, a surfactant to form an organic phase;
C.d) optionally heating the organic phase;
C.e) dispensing the organic phase into an aqueous mixture comprising at least one hydrophilic polymer, and one or more hydrophilic drug(s) while stirring to form a microemulsion; and
C.f) stabilising the polymer-lipid microemulsion by adding a phosphate buffer at 0° C. while stirring.

38. The process according to claim 37, further comprising, at step a), dissolving PLGA, or alternatively, any biocompatible and biodegradable polymer suitable for use in active compound delivery, including polylactic acid, polyglycolic acid, or poly ϵ-caprolactone, into the polar aprotic solvent with the fatty acid.

39. The process according to claim 37 or 38, further comprising, at step c), adding drop-wise, the organic carboxylic acid with the surfactant.

40. The process according to any one of claims 37 to 39, further comprising in step e) heating at from between about 40° C. to 50° C. while stirring to form the microemulsion.

41. The process according to any one of claims 37 to 40, wherein the phosphate buffer comprises a pH of from about 7.2 to about 7.6 at 0° C.

42. The process according to claim 41, wherein the phosphate buffer pH is about 7.4 at 0° C.

43. The process according to any one of claims 37 to 42, wherein stabilisation of the microemulsion is performed by adding the microemulsion to the phosphate buffer solution at a ratio about 1:1.

44. The process according to claim 25, wherein the freeze drying is performed following an initial snap-freezing step in liquid nitrogen.

45. A method for the treatment or inhibition of viral ARDS with the polymer-lipid microemulsion delivery system described in any one of claims 1 to 18, comprising one or more drug(s) selected from the group consisting of antiviral drug(s); immunomodulatory compound(s); and antiviral lectin(s).

46. The method according to claim 45, wherein the viral ARDS is influenza or SARS-CoV, including SARS-CoV-2 and MERS-CoV.

47. The method according to claim 46, wherein the viral ARDS is SARS-CoV-2.

48. The method according to any one of claims 45 to 47, comprising delivery by pulmonary administration of a liquid formulation of the polymer-lipid microemulsion delivery system as described in any one of claims 1 to 18.

49. The method according to any one of claims 45 to 47, comprising delivery by oral or intravenous administration of a powder formulation of the polymer-lipid microemulsion delivery system as described in any one of claims 1 to 18.

50. The method according to any one of claims 45 to 49, comprising simultaneous delivery by pulmonary administration of a liquid formulation of the polymer-lipid microemulsion delivery system as described in any one of claims 1 to 18, and oral or intravenous administration of a powder formulation of the polymer-lipid microemulsion delivery system as described in any one of claims 1 to 18.

51. The method according to any one of claims 45 to 50, comprising a step of nebulising the liquid polymer-lipid microemulsion delivery system for delivery by inhalation, including for pulmonary delivery.
